(19) United States
(12) Patent Application Publication
(10) Pub. No.: US 2021/0113696 A1
(43) Pub. Date: Apr. 22, 2021

(54) IONIC LIQUID COMPOSITIONS FOR TREATMENT OF ROSACEA

(71) Applicant: CAGE BIO INC., San Carlos, CA (US)

(72) Inventors: Nitin JOSHI, San Carlos, CA (US); Keith HALL, San Carlos, CA (US); Victor O. NAVA-SELGADO, San Carlos, CA (US); Marina SHEVACHMAN, San Carlos, CA (US)

(21) Appl. No.: 16/981,956

(22) PCT Filed: Mar. 19, 2019
(86) PCT No.: PCT/US2019/023032

§ 371 Date: Sep. 17, 2020

Related U.S. Application Data
(60) Provisional application No. 62/644,921, filed on Mar. 19, 2018.
(60) Provisional application No. 62/800,280, filed on Feb. 1, 2019.

(51) Int. Cl. A61K 47/14; A61K 9/08; A61K 31/14; A61K 31/201; A61K 47/38; A61K 9/00; A61K 45/06; A61K 9/06; A61P 17/00; A61K 47/10; A61K 47/06; A61K 47/12

(52) U.S. Cl. CPC A61K 47/14; A61K 9/08; A61K 31/14; A61K 31/201; A61K 47/38; A61K 47/12; A61K 45/06; A61K 9/06; A61P 17/00; A61K 47/10; A61K 47/06; A61K 9/0014

(57) ABSTRACT

Disclosed herein, in certain embodiments, are anti-microbial and anti-inflammatory pharmaceutical compositions comprising an ionic liquid comprising a choline cation and fatty acid anion for use in the treatment of rosacea. In some embodiments, the ionic liquid is further formulated in a solvent or a gel base for topical administration.

### CROSS-REFERENCE

This application claims the benefit of U.S. Provisional Application No. 62/800,280, filed on Feb. 1, 2019 and U.S. Provisional Application No. 62/644,921, filed on Mar. 19, 2018, each of which is incorporated herein by reference in its entirety.

### BACKGROUND OF THE DISCLOSURE

Rosacea is a common inflammatory skin disorder affecting over 15 million people worldwide. The primary symptoms of rosacea are erythema (abnormal redness of the skin), telangiectasia (visible red lines due to abnormal dilation of capillary vessels), pimple-like eruptions (papules) and pustules. Currently available topical treatments have limited effectiveness and cannot treat all symptoms, particularly erythema. Surgery, such as the laser elimination of blood vessels, is typically a last resort, but may be prescribed if other treatments are ineffective.

### SUMMARY OF THE DISCLOSURE

Disclosed herein, in certain embodiments, are methods for treating a disease or a condition related to rosacea in an individual in need thereof, comprising administering to a skin of the individual a composition comprising: (a) an ionic liquid comprising a choline cation and geranic acid anion; and (b) a pharmaceutically acceptable solvent. In some embodiments, the pharmaceutically acceptable solvent is selected from the group consisting of: water, ethanol, diisopropyl adipate, polyethylene glycol (PEG), glycerin, propylene glycol, and a combination thereof. In some embodiments, the composition further comprises a gelling agent. In some embodiments, the gelling agent is selected from the group consisting of: hydroxyethyl cellulose (HEC), hydroxypropyl cellulose (HPC), hydroxypropylmethyl cellulose (HPMC), and a combination thereof.

In some embodiments, the ionic liquid comprises the choline cation and geranic acid anion in a molar ratio of 1:1 or 1:2 of choline cation to geranic acid anion. In some embodiments, the ionic liquid comprises the choline cation and geranic acid anion in a molar ratio in a range of 1:1 to 1:4 of choline cation to geranic acid anion. In some embodiments, the ionic liquid comprises the choline cation and geranic acid anion in a molar ratio of 1:1, 1:2, 1:3, or 1:4 of choline cation to geranic acid anion. In some embodiments, the composition provides an increased antimicrobial action compared to an antimicrobial action of choline or an antimicrobial action of geranic acid. In some embodiments, the increased antimicrobial action is a 10 fold less concentration of the composition required for complete killing of a microbe relative to a concentration of choline or a concentration of geranic acid required for complete killing of the microbe. In some embodiments, the composition provides an increased skin permeation relative to a skin permeation of choline or a skin permeation of geranic acid. In some embodiments, the composition provides an increased conductivity relative to a conductivity of geranic acid and a decreased conductivity relative to a conductivity of choline. In some embodiments, the ionic liquid comprises a concentration of about 0.1% to 99% of the composition, and the pharmaceutically acceptable solvent comprises a concentration of about 1% to about 99.9% of the composition.

In some embodiments, the composition is formulated for transdermal administration. In some embodiments, the composition further comprises an additional therapeutic agent selected from the group consisting of: a small molecule drug, an antimicrobial agent, a protein, a peptide, an antibody, a nucleic acid, a chemotherapy agent, and a combination thereof. In some embodiments, the composition is formulated as a gel, lotion, cream, ointment, solution, or a patch. In some embodiments, erythema of the skin of the individual is reduced. In some embodiments, inflammation of the skin of the individual is reduced. In some embodiments, lesions on the skin of the individual are reduced.

In some embodiments, the composition further comprises a fragrance agent. In some embodiments, the fragrance agent is an acid or a terpene of a citrus fruit. In some embodiments, the citrus fruit is an orange, a grapefruit, a lime, or a lemon. In some embodiments, the terpene is D-limonene. In some embodiments, the acid is citric acid or a derivative thereof.

Disclosed herein, in certain embodiments, are methods for treating an inflammatory or infectious skin disease or condition in an individual in need thereof, comprising administering to a skin of the individual a composition comprising: (a) an ionic liquid comprising a choline cation and a geranic acid anion; and (b) a pharmaceutically acceptable solvent selected from the group consisting of: diisopropyl adipate, polyethylene glycol (PEG), glycerin, propylene glycol, and a combination thereof. Further disclosed herein, in certain embodiments, are methods for treating a skin condition in an individual in need thereof, comprising administering to a skin of the individual a composition comprising: (a) an ionic liquid comprising a choline cation and a geranic acid anion; (b) a pharmaceutically acceptable solvent selected from the group consisting of: water, ethanol, diisopropyl adipate, polyethylene glycol (PEG), glycerin, propylene glycol, and a combination thereof; and (c) a gelling agent.

In some embodiments, the inflammatory or infectious skin disease or condition is rosacea, molluscum contagiosum, or onychomycosis. In some embodiments, the inflammatory or infectious skin disease or condition is rosacea. In some embodiments, the inflammatory or infectious skin disease or condition is molluscum contagiosum. In some embodiments, the inflammatory or infectious skin disease or condition is onychomycosis. In some embodiments, the skin disease or condition is an inflammatory skin disease or condition. In some embodiments, the skin condition is caused by a mite, bacteria, or a combination thereof. In some embodiments, the composition does not induce development of resistance in the mite or the bacteria. In some embodiments, erythema of the skin of the individual is reduced. In some embodiments, inflammation of the skin of the individual is reduced. In some embodiments, lesions on the skin of the individual are reduced. In some embodiments, redness on the skin is reduced.

In some embodiments, the ionic liquid comprises the choline cation and geranic acid anion in a molar ratio of 1:1 or 1:2 of choline cation to geranic acid anion. In some embodiments, the ionic liquid comprises the choline cation and geranic acid anion in a molar ratio in a range of 1:1 to 1:4 of choline cation to geranic acid anion. In some embodiments, the ionic liquid comprises the choline cation and geranic acid anion in a molar ratio of 1:1, 1:2, 1:3, or 1:4 of choline cation to geranic acid anion. In some embodiments, the composition provides an increased antimicrobial action compared to an antimicrobial action of choline or an antimicrobial action of geranic acid. In some embodiments, the increased antimicrobial action is a 10 fold less concentration of the composition required for complete killing of a microbe relative to a concentration of choline or a concentration of geranic acid required for complete killing of the microbe. In some embodiments, the composition provides an increased skin permeation relative to a skin permeation of choline or a skin permeation of geranic acid. In some embodiments, the composition provides an increased conductivity relative to a conductivity of geranic acid and a decreased conductivity relative to a conductivity of choline. In some embodiments, the ionic liquid comprises a concentration of about 0.1% to 99% of the composition, and the pharmaceutically acceptable solvent comprises a concentration of about 1% to about 99.9% of the composition.

In some embodiments, the composition is formulated for transdermal administration. In some embodiments, the composition further comprises an additional therapeutic agent selected from the group consisting of: a small molecule drug, an antimicrobial agent, a protein, a peptide, an antibody, a nucleic acid, a chemotherapy agent, and a combination thereof. In some embodiments, the composition is formulated as a gel, lotion, cream, ointment, solution, or a patch. In some embodiments, the gelling agent is selected from the group consisting of: hydroxyethyl cellulose (HEC), hydroxypropyl cellulose (HPC), hydroxypropylmethyl cellulose (HPMC), and a combination thereof.

Disclosed herein, in certain embodiments, are compositions comprising: (a) an ionic liquid comprising a choline cation and a geranic acid anion; and (b) a pharmaceutically acceptable solvent selected from the group consisting of: diisopropyl adipate, polyethylene glycol (PEG), glycerin, propylene glycol, and a combination thereof. Further disclosed herein, in certain embodiments, are compositions comprising: (a) an ionic liquid comprising a choline cation and a geranic acid anion; (b) a pharmaceutically acceptable solvent selected from the group consisting of: water, ethanol, diisopropyl adipate, polyethylene glycol (PEG), glycerin, propylene glycol, and a combination thereof; and (c) a gelling agent.

In some embodiments, the ionic liquid comprises the choline cation and geranic acid anion in a molar ratio of 1:1 or 1:2 of choline cation to geranic acid anion. In some embodiments, the ionic liquid comprises the choline cation and geranic acid anion in a molar ratio in a range of 1:1 to 1:4 of choline cation to geranic acid anion. In some embodiments, the ionic liquid comprises the choline cation and geranic acid anion in a molar ratio of 1:1, 1:2, 1:3, or 1:4 of choline cation to geranic acid anion. In some embodiments, the composition provides an increased antimicrobial action compared to an antimicrobial action of choline or an antimicrobial action of geranic acid. In some embodiments, the increased antimicrobial action is a 10 fold less concentration of the composition required for complete killing of a microbe relative to a concentration of choline or a concentration of geranic acid required for complete killing of the microbe. In some embodiments, the composition provides an increased skin permeation relative to a skin permeation of choline or a skin permeation of geranic acid. In some embodiments, the composition provides an increased conductivity relative to a conductivity of geranic acid and a decreased conductivity relative to a conductivity of choline. In some embodiments, the ionic liquid comprises a concentration of about 0.1% to 99% of the composition, and the pharmaceutically acceptable solvent has a concentration of about 1% to about 99.9% of the composition.

In some embodiments, the composition is formulated for transdermal administration. In some embodiments, the composition further comprises an additional therapeutic agent selected from the group consisting of: a small molecule drug, an antimicrobial agent, a protein, a peptide, an antibody, a nucleic acid, a chemotherapy agent, and a combination thereof. In some embodiments, the composition is formulated as a gel, lotion, cream, ointment, solution, or a patch. In some embodiments, the gelling agent is selected from the group consisting of: hydroxyethyl cellulose (HEC), hydroxypropyl cellulose (HPC), hydroxypropylmethyl cellulose (HPMC), and a combination thereof.

In some embodiments, the composition comprises 20% to 60% of an ionic liquid comprising a choline cation and a geranic acid anion, 5% to 20% propylene glycol, and a remaining balance of water. In some embodiments, the composition comprises 30% to 50% of the ionic liquid. In some embodiments, the composition comprises a molar ratio of the choline cation and geranic acid anion of 1:2. In some embodiments, the composition comprises 10% to 15% propylene glycol. In some embodiments, the composition further comprises 0.5% to 5% hydroxyethyl cellulose. In some embodiments, the composition further comprises 0.5% to 5% D-limonene. In some embodiments, the composition is formulated as a gel. In some embodiments, the composition is formulated for topical administration. In some embodiments, the composition is formulated for twice daily administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 ¹H NMR spectra data of Composition B.

FIG. 2 ¹H NMR spectra data of Composition A.

FIG. 3 illustrates conductivity of geranic acid and propylene glycol in varying concentrations of % propylene glycol.

FIG. 4 illustrates conductivity of choline and propylene glycol in varying concentrations of % propylene glycol.

FIG. 5 illustrates conductivity of choline vs. Composition A in varying concentrations of % propylene glycol.

FIG. 6 illustrates conductivity of geranic acid in varying concentrations of % ethanol.

FIG. 7 illustrates conductivity of choline in varying concentrations of % ethanol.

FIG. 8 illustrates conductivity of choline vs. Composition A in varying concentrations of % ethanol.

FIG. 9 illustrates conductivity of choline in propylene glycol to the conductivity of choline in ethanol.

FIG. 10 illustrates conductivity of Composition A in varying concentrations of % water.

FIG. 11 illustrates conductivity of Composition A in varying concentrations of % propylene glycol.

FIG. 12 illustrates conductivity of Composition A in varying concentrations of % glycerin.

FIG. 13 illustrates conductivity of Composition A in varying concentrations of % PEG400.

FIG. 14 illustrates conductivity of Composition A in varying concentrations of % ethanol.

FIG. 15 illustrates conductivity of Composition A in varying concentrations of % diisopropyl adipate.

FIG. 16 illustrates conductivity of propylene glycol, ethanol, Composition A, and water with increasing amounts of % water.

FIG. 17 illustrates conductivity of glycerin, PEG400, Composition A, and water with increasing concentrations of % water.

FIG. 18 illustrates cumulative flux of choline in Composition A.

FIG. 19 illustrates average flux of choline in Composition A.

FIG. 20 illustrates cumulative flux of geranic acid in Composition A.

FIG. 21 illustrates average flux of geranic acid in Composition A.

FIG. 22 illustrates cumulative flux of choline in Composition A compared to Composition B in water or diisopropyl adipate (DIA).

FIG. 23 illustrates average flux of choline in Composition A compared to Composition B in water or diisopropyl adipate (DIA).

FIG. 24 illustrates cumulative flux of geranic acid in Composition A compared to Composition B in water or diisopropyl adipate (DIA).

FIG. 25 illustrates average flux of geranic acid in Composition A compared to Composition B in water or diisopropyl adipate (DIA).

FIG. 26 illustrates conductivity of an ionic liquid comprising choline cations:geranic acid anions in a 1:2 molar ratio (where water was not evaporated off as it was with Composition A) in varying concentrations of % of a gel base comprising glycerin, diisopropyl adipate, ethanol, and HPC.

FIG. 27 illustrates conductivity of Composition A in varying concentrations of % of a gel base comprising glycerin, diisopropyl adipate, ethanol, and HPC.

FIG. 28 illustrates conductivity of Composition A in varying concentrations of % of a gel base comprising propylene glycol, diisopropyl adipate, ethanol, and HPC.

FIG. 29 illustrates conductivity of Composition A in varying concentrations of % of a gel base comprising diisopropyl adipate, ethanol, HPC, and either propylene glycol or glycerin.

FIG. 30 illustrates the accumulated dose of choline over time (in μg). A Dixon's Qtest with 95% confidence was first run on the data to identify and remove outliers.

FIG. 31A illustrates the accumulated dose of geranic acid over time (in μg). A Dixon's Qtest with 95% confidence was first run on the data to identify and remove outliers.

FIG. 31B illustrates the amount delivered (microgram, y-axis) over time of geranic acid anions (black bars) and choline cations (white bars).

FIG. 32 illustrates conductivity of Composition B in varying concentrations of % water.

FIG. 33 illustrates average lesion count (y-axis) over time (weeks, x-axis) following administration of Composition A.

FIG. 34 illustrates percent reduction from baseline (y-axis) over time (weeks, x-axis) following administration of Composition A.

FIG. 35 illustrates number of patients (y-axis) having clear (hatched bars), almost clear (dotted bars), mild (horizontal bars), and moderate (black bars) skin as measured by Investigator's Global Assessment at each visit (x-axis).

FIG. 36 illustrates number of patients (y-axis) having clear (hatched bars), almost clear (dotted bars), mild (horizontal bars), moderate (black bars), and severe (checkered bars) redness as measured Investigator's Global Assessment of Redness at each visit (x-axis).

FIG. 37 illustrates comparison of Composition A to a Comparator in improving symptoms of rosacea. Percent reduction in lesion count at 2 weeks (first set of bars from left) and 4 weeks (second set of bars from left) were measured following administration of Composition A (black bars) or Comparator in two studies (white bars and vertical bars). Percent of patients with almost clear or clear skin measured by Investigator's Global Assessment at 2 weeks (third set of bars from left) and 4 weeks (fourth set of bars from left) were measured following administration of Composition A (black bars) or Comparator in two studies (white bars and vertical bars).

### DETAILED DESCRIPTION OF THE DISCLOSURE

Described herein, in certain embodiments, are compositions and methods for treating an inflammatory or infectious skin disease or condition in an individual in need thereof. In some embodiments, the skin condition is rosacea. In some embodiments, the method comprises administering to a skin of the individual a composition comprising an ionic liquid and a pharmaceutically acceptable solvent. In some embodiments the ionic liquid comprises a cation and an anion. In some embodiments, the ionic liquid comprises a choline cation and a fatty acid anion. In some embodiments, the fatty acid anion is a geranic acid anion. In some embodiments, the cation has anti-inflammatory properties. In some embodiments, the anion has anti-microbial properties. In some embodiments, the pharmaceutically acceptable solvent is water, ethanol, diisopropyl adipate, polyethylene glycol (PEG), glycerin, propylene glycol, and a combination thereof. In some embodiments, the composition further comprises a gelling agent.

### Ionic Liquid Compositions

Described herein, in certain embodiments, are compositions comprising an ionic liquid comprising a choline cation and a fatty acid anion. In some embodiments, the composition further comprises a pharmaceutically acceptable solvent. In some embodiments, the fatty acid is myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is geranic acid. In some embodiments, the fatty acid comprises 9 to 14 carbons. In some embodiments, the ionic liquid is liquid at room temperature. In some embodiments, the ionic liquid is liquid below 100° C.

In some embodiments, the ionic liquid is a deep eutectic solvent (DES). In some embodiments, a DES comprises excess carboxylate which precludes 1:1 ion pairing. In some embodiments, a DES further comprises a hydrogen-bond donor. In some embodiments, the hydrogen-bond donor is urea or citric acid. In some embodiments, the solvent properties of a DES are adjusted by changing the hydrogen-bond donor. In some embodiments, the ammonium salt of a DES interacts with a hydrogen-bond donor. In some embodiments, the DES has a melting point lower than either of the individual components (e.g. fatty acid and choline).

In some embodiments, the ionic liquid comprises a molar ratio of a choline cation to a fatty acid anion of 1:0.5 to 1:10. In some embodiments, the molar ratio of the choline cation to the fatty acid anion is about 1:0.5, 1:0.6, 1:0.7, 1:0.8, 1:0.9, 1:1.0; 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9. 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, or about 1:10. In some embodiments, the molar ratio of the choline cation to the fatty acid anion is about 1:1.1, 1:1.2, 1:1.3, 1:1.4, 1:1.5, 1:1.6, 1:1.7, 1:1.8, 1:1.9, or 1:2.0.

In some embodiments, the choline cation and fatty acid anion are in a molar ratio in the ionic liquid. In some embodiments, the choline cation and fatty acid anion are in a molar ratio of 1:1. In some embodiments, the term Composition B is used herein to refer to a composition or an ionic liquid comprising a 1:1 molar ratio of choline cation to geranic acid anion. In some embodiments, Composition B does not comprise water.

In other embodiments, the choline cation and fatty acid anion are in a molar ratio of 1:2. In some embodiments, the term Composition A is used herein to refer to a composition or an ionic liquid comprising a 1:2 molar ratio of choline cation to geranic acid anion. In some embodiments, Composition A does not comprise water.

In some embodiments, the chemical structure of choline is:

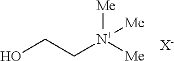

wherein X⁻ is a pharmaceutically acceptable anion.

In some embodiments, term choline refers to the class of quaternary ammonium salts containing the N,N,N-trimethylethanolammonium cation. In some embodiments, the X⁻ on the right of the structure of choline denotes a pharmaceutically acceptable anion. In some embodiments the X⁻ is bicarbonate, carbonate, acetate, citrate, tartarate, bitartarate, lactate, chloride, bromide, or iodide. In some embodiments, the X⁻ is bicarbonate. In some embodiments, the choline is an anti-inflammatory agent.

In some embodiments, choline is in the form of a pharmaceutically acceptable salt. The type of pharmaceutical acceptable salts, include, but are not limited to acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some embodiments, the chemical structure of geranic acid, or 3,7-dimethyl-2,6-octadienoic acid, is:

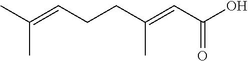

In some embodiments, geranic acid is in the form of a pharmaceutically acceptable salt. The type of pharmaceutical acceptable salts, include, but are not limited to salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Examples of acceptable organic bases include, but are not limited to, ethanolamine, diethanolamine, triethanolamine, tromethamine, and N-methylglucamine. Examples of acceptable inorganic bases include, but are not limited to, aluminum hydroxide, calcium hydroxide, potassium hydroxide, sodium carbonate, and sodium hydroxide.

In some embodiments, the choline and the fatty acid are synthesized using any suitable standard synthetic reactions. In some embodiments, the reactions are employed in a linear sequence to provide the compounds or they may be used to synthesize fragments which are subsequently joined by any suitable method. In some embodiments, the starting material used for the synthesis of choline or fatty acid is synthesized or are obtained from commercial sources.

In some embodiments, geranic acid is purified from the commercially available technical grade (Sigma-Aldrich, St. Louis, Mo.) by repeated (5-7×) recrystallization from a solution of 70 wt % geranic acid/30 wt % acetone at −70° C. In some embodiments, purity of the geranic acid is assessed by ¹H NMR spectroscopy and conductivity measurements. In some embodiments, the term geranic acid refers to a geranic acid or a salt thereof. In some embodiments, the geranic acid is an anti-microbial agent.

In some embodiments, the pharmaceutically acceptable solvent is water, ethanol, diisopropyl adipate, polyethylene glycol (PEG), glycerin, propylene glycol, a short chain fatty acid, a fatty acid ester, or a combination thereof. In some embodiments, the pharmaceutically acceptable solvent is a liquid alcohol, liquid glycol, liquid polyalkalene glycol, liquid ester, liquid amine, liquid protein hydrolysate, liquid alkalated protein hydrolysate, liquid lanolin, lanolin derivative, or water. In some embodiments, the pharmaceutically acceptable solvent is diisopropyl adipate. In some embodiments, the composition is miscible with the pharmaceutically acceptable solvent. In some embodiments, at least one of the individual components of the composition is not miscible with pharmaceutically acceptable solvent. In some embodiments, the composition is miscible with diisopropyl adipate. In some embodiments, at least one of the individual components of the composition is not miscible with diisopropyul adipate. In some embodiments, the water is deionized water or Milli-Q® water. In some embodiments, the composition does not comprise a preservative. Examples of preservatives include, but are not limited to, a paraben or a phenoxyethanol.

In some embodiments, the composition comprises an increased antimicrobial action compared to an antimicrobial action of choline or an antimicrobial action of the fatty acid. In some embodiments, the increased antimicrobial action is a 10 fold less concentration of the composition required for complete killing of a microbe relative to a concentration of choline or a concentration of the fatty acid required for complete killing of the microbe.

In some embodiments, the composition comprises an increased skin permeation (or permeability) relative to a skin permeation of choline or a skin permeation of the fatty acid. In some embodiments, the composition increases skin permeation by disrupting the stratum corneum lipids, interacting with the intercellular proteins, improving portioning of the drug into the lipid layers, or a combination thereof. In some embodiments, the composition penetrates into the epidermis and dermis. In some embodiments, the composition penetrates into a nail substrate. In some embodiments, the nail substrate comprises the nail plate, the nail matrix, the nail bed, or a combination thereof. In some embodiments, the composition achieves an Effect Site Concentration (Cₑₛ) in the dermis greater than the minimal inhibitory concentration (MIC) of the anti-microbial agent. In some embodiments, the anti-microbial agent is the fatty acid anion.

In some embodiments, the composition enhances delivery of small molecules, large molecules, or a combination thereof, through the skin. In some embodiments, small molecules have a molecular weight of less than 500 Da. In some embodiments, large molecules have a molecular weight of up to 150 kDa.

In some embodiments, the composition has decreased skin irritation relative to a skin irritation of choline or a skin irritation of the fatty acid. In some embodiments, the composition exhibits minimal cytotoxicity relative to a cytotoxicity of choline or a cytotoxicity of the fatty acid. In some embodiments, the composition comprises an increased conductivity relative to a conductivity of the fatty acid and a decreased conductivity relative to a conductivity of choline.

In some embodiments, the composition is clear. In some embodiments, the composition is turbid. In some embodiments, the composition is opaque. In some embodiments, the composition is yellow. In some embodiments, the composition is a colloidal system.

In some embodiments, the composition is formulated for transdermal administration. In some embodiments, the composition is formulated as a gel, lotion, cream, ointment, solution, or a patch. In some embodiments, the composition is formulated as a gel. In some embodiments, the patch is an adhesive-based patch or a reservoir-based patch. In some embodiments, the patch is a hypoallergenic patch.

In some embodiments, the composition further comprises a gelling agent, a viscosity modifying agent, or a combination thereof. In some embodiments, the gelling agent or the viscosity modifying agent is also a bulking agent.

Examples of gelling agents or viscosity modifying agents include, but are not limited to, as polyvinyl alcohol, polyethylene oxide, different poloxamers, carbopols, or celluloses such as ethyl cellulose, hydroxyl ethyl cellulose, hydroxyl propyl cellulose, hydroxyl propyl methyl cellulose, sulfoxides or similar compounds such as dimethylsulfoxide, dimethylsulfoxide, dimethylacetamide, dimethylformamide, pyrrolidones such as 2-pyrrolidone, N-methyl-2-pyrrolidone, 1-lauryl-2-pyrrolidone, alcohols such as ethanol, 1-octanol, 1-hexanol, 1-decanol, lauryl alcohol, linolenyl alcohol, glycols such as propylene glycol, butane-1,2-diol, polyethylene glycol 400, urea and derivatives urea, such as 1-dodecylurea, 1-dodecyl-3-methylurea, 1-dodecyl-3-methylthiourea, azone and azone like molecules such as (laurocapram; 1-dodecylazacycloheptan-2-one), 1-alkyl- or 1-alkenylazacycloalkanones, enzymes acid phosphatase, calonase, papain Iminosulfuranes S, S-dimethyl-N-(5-nitro-2-pyridyl) iminosulfurane, S, S-dimethyl-N-(4-bromobenzoyl) iminosulfurane, cyclodextrins 2-hydroxypropyl-β-cyclodextrin, methylated-β-cyclodextrin, fatty acid esters such as cetyl lactate, butylacetate, isopropyl myristate Fatty acids alkanoic acids, oleic acid, lauric acid, capric acid, surfactants such as sorbitan monopalmitate, sorbitan trioleate, cetyl trimethyl ammonium bromide, sodium lauryl sulfate, terpenes such as limonene, nerolidol, farnesol, carvone, menthone, polymers such as β-D-glucopyranosyl-terminated oligodimethylsiloxanes, 1-alkyl-3-β-D-glucopyranosyl-1,1,3,3-tetramethyldisiloxanes Monoolein monoolein Oxazolidinones 4-decyloxazolidin-2-one, 3-acetyl-4-decyloxazolidin-2-one, carbomer, methyl cellulose, sodium carboxyl methyl cellulose, carrageenan, colloidal silicon dioxide, guar gum, gelatin, alginic acid, sodium alginate, and fumed silica. In some embodiments, the gelling agent is a hydroxyethyl cellulose (HEC), hydroxypropyl cellulose (HPC), hydroxypropylmethyl cellulose (HPMC), or a combination thereof. In some embodiments, the gelling agent is HPC.

In some embodiments, the combination of a gelling agent and a pharmaceutically acceptable solvent is referred to as a gel base. In some embodiments, a gel base is created prior to the addition of an ionic liquid to the gel base. In some embodiments, the ionic liquid is added into the gel base. In some embodiments, the gel base is added into the ionic liquid.

In some embodiments, the gel base comprises water and a gelling agent. In some embodiments, the gel base comprises diisopropyl adipate and a gelling agent. In some embodiments, the gel base comprises PEG400 and a gelling agent. In some embodiments, the gel base comprises propylene glycol and a gelling agent. In some embodiments, the gelling agent is HEC, HPC, or HPMC. In some embodiments, the gel base comprises ethanol and a gelling agent. In some embodiments, the gel base further comprises glycerin, propylene glycol, ethanol, or a combination thereof.

In one example, the gel base comprises diisopropyl adipate, ethanol, glycerin, and HPC. In some embodiments, the gel base comprises 25% w/w of diisopropyl adipate, 43% w/w ethanol, 30% w/w glycerin, and 3% w/w HPC. In another example, the gel base comprises diisopropyl adipate, ethanol, propylene glycol, and HPC. In some embodiments, the gel base comprises 25% w/w of diisopropyl adipate, 13% w/w ethanol, 60% w/w propylene glycol, and 3% w/w HPC.

In some embodiments, a composition comprises a bulking agent with a concentration from 1 to 10%. In some embodiments, a composition comprises a gelling agent with a concentration from 1 to 10%.

In some embodiments, the composition comprises an additional therapeutic agent selected from the group consisting of: a small molecule drug, an antimicrobial agent, a protein, a peptide, an antibody, a nucleic acid, a chemotherapy agent, and a combination thereof.

In some embodiments, the small molecule drug is a beta blocker, a loop diuretic, crotamiton, a retinoid, oxymetazoline hydrochloride, brimonidine, benzoyl peroxide, or a Janus kinase (JAK) inhibitor. In some embodiments, the beta blocker is propranolol, sotalol, atenolol, metoprolol, bisoprolol, carvedilol, nebivolol, or labetalol. In some embodiments, the loop diuretic is furosemide, bumetanide, or torsemide. In some embodiments, the small molecule drug is a vasodilator. In some embodiments, the retinoid is isotretinoin or adapalene. In some embodiments, the JAK inhibitor is tofacitinib, ocalcitinib, or ruxolitinib. In some embodiments, the small molecule drug is a prostacyclin analog. In some embodiments, the prostacyclin analog is treprostinil, epoprostenol, or iloprost. In some embodiments, the protein is insulin or albumin. In some embodiments, the composition comprises about 3.5 mg/mL of insulin. In some embodiments, the peptide is a dekapeptide. In some embodiments, the dekapeptide stimulates matrix regeneration, modulates melanin synthesis, stimulates lipolysis, deregulates cytokine release, or a combination thereof. In some embodiments, the chemotherapy agent is paclitaxel. In some embodiments, the composition comprises about 400 mg/mL of paclitaxel. In some embodiments, the nucleic acid is a small interfering RNA (siRNA) or a microRNA (miRNA). In some embodiments, the antimicrobial agent is a benzalkonium chloride, benzyl benzoate, sodium sulfacetamide, metronidazole, diaminodiphenyl sulfone (DDS; dapsone), permethrin, ivermectin, erythromycin, clindamycin, or azelaic acid. In some embodiments, the antimicrobial agent is an anti-acaride, anti-bacterial, anti-viral, anti-yeast, or anti-fungal agent. In some embodiments, the additional therapeutic agent is tea tree oil.

In some embodiments, the additional therapeutic agent is delivered into systemic circulation. In some embodiments, the additional therapeutic agent has low solubility.

In some embodiments, the composition further comprises a non-ionic surfactant. In some embodiments, the non-ionic surfactant is poloxamer or polysorbate 80. In some embodiments, the poloxamer is a Pluronic®, Kollipho®, or Synperonic®. In some embodiments, the non-ionic surfactant comprises a concentration in the composition ranging from 0.1 to 20%.

In some embodiments, the composition further comprises an inactive ingredient. In some embodiments, the inactive ingredient enhances long-term shelf storage or target area absorption. In some embodiments, the inactive ingredient is an emollient/stiffening agents/ointment, an emulsifying agent/solubilizing agent, a humectant, a preservative, a permeation enhancer, a chelating agent, an antioxidant, vehicles/solvents, pH adjusting agents, or a combination thereof.

Example of emollients/stiffening agents/ointments include, but are not limited to, carnauba wax, cetyl alcohol, cetostearyl alcohol, cetyl ester wax, emulsifying wax, hydrous lanolin, lanolin, lanolin alcohols, microcrystalline wax, paraffin, petrolatum, polyethylene glycol and polymers thereof, stearic acid, stearyl alcohol, white wax, and yellow wax. Examples of emulsifying agents/solubilizing agents include, but are not limited to, glyceryl monostearate, glyceryl monooleate, glyceryl isostearate, polysorbate 20, polysorbate 80, polysorbate 60, poloxamer, emulsifying wax, sorbitan monostearate, sorbitan monooleate, sodium lauryl sulfate, propylene glycol monostearate, diethylene glycol monoethyl ether, and docusate sodium. Examples of humectants include, but are not limited to, glycerin, propylene glycol, polyethylene glycol, sorbitol solution, and 1,2,6-hexanetriol. Examples of preservatives include, but are not limited to, benzoic acid, propyl paraben, methyl paraben, imidurea, sorbic acid, potassium sorbate, benzalkonium chloride, phenyl mercuric acetate, chlorobutanol, and phenoxyethanol. Examples of permeation enhances include, but are not limited to, propylene glycol, ethanol, isopropyl alcohol, oleic acid, and polyethylene glycol. Examples of chelating agents include, but are not limited to, ethylene diamine tetraacetate. Examples of antioxidants include, but are not limited to butylated hydroxyanisole and butylated hydroxytoluene. Examples of vehicles/solvents include, but are not limited to purified water, hexylene glycol, propylene glycol, oleyl alcohol, propylene carbonate, mineral oil, ethanol, diisopropyl adipate, polyethylene glycol (PEG), and glycerin. Examples of pH adjusting agents include, but are not limited to, acids such as acetic, boric, citric, lactic, phosphoric and hydrochloric acids; and bases such as sodium hydroxide, sodium phosphate, sodium borate, sodium citrate, sodium acetate, sodium bicarbonate, sodium lactate, ammonium chloride, and tris-hydroxymethylaminomethane. In some embodiments, the composition further comprises trolamine.

In some embodiments, the inactive ingredient is an acrylate or polymer thereof, methacrylate or polymer thereof, cellulose polymer, hydroxyethyl cellulose or polymer thereof, poly-lactylate polymer, polyvinyl pyrrolidone polymer, ethylenevinylacetate copolymer, short, medium and long chain fatty acid molecules or analog thereof, isopropryl myristate, polyethylene terephthalate, vitamin C, vitamin C analog or ester, vitamin E, vitamin E analog, vitamin E polymeric compound, d-α-tocopheryl polyethylene glycol 1000 succinate (vitamin E TPGS), or silicone.

In some embodiments, the inactive ingredient comprises dual or multiple functionalities. For example, in one embodiment, polyethylene glycol is an emollient, humectant, and a permeation enhancer.

In some embodiments, each component in a composition, such as the ionic liquid, the pharmaceutically acceptable solvent, and optionally other components, is described a percent (%) of the composition. In some embodiments, the % of the composition is a percent concentration volume/volume (v/v) or a percent concentration weight/volume (w/v).

In some embodiments, the composition comprises the ionic liquid in a concentration of about 0.1% to 99%. In some embodiments, the composition comprises the ionic liquid in a concentration of about 1% to 40%. In some embodiments, the composition comprises the ionic liquid in a concentration of about 1% to 20%. In some embodiments, the composition comprises the ionic liquid in a concentration of about 5% to 20%. In some embodiments, the composition comprises the ionic liquid in a concentration of about 5% to 40%. In some embodiments, the composition comprises the ionic liquid in a concentration of about 20% to 40%. In some embodiments, the composition comprises the ionic liquid in a concentration of about 20% to 60%. In some embodiments, the composition comprises the ionic liquid in a concentration of about 20% to 80%.

In some embodiments, the composition comprises the ionic liquid in a concentration of about 0.1% to 99%, and the pharmaceutically acceptable solvent in a concentration of about 1% to about 99.9%. In some embodiments, the composition comprises the ionic liquid in a concentration of about 1% to 40%, and the pharmaceutically acceptable solvent in a concentration of about 60% to about 99%. In some embodiments, the composition comprises the ionic liquid in a concentration of about 20% to 40%, and the pharmaceutically acceptable solvent in a concentration of about 80% to about 99%. In some embodiments, the composition comprises the ionic liquid in a concentration of about 20% and the pharmaceutically acceptable solvent in a concentration of about 80%. In some embodiments, the composition comprises the ionic liquid in a concentration of about 40% and the pharmaceutically acceptable solvent in a concentration of about 60%.

In some embodiments, the composition further comprises ethanol. In some embodiments, the concentration of ethanol in the composition is about 1%, 5%, 10%, 20%, 30%, 40%, or 50%.

In some embodiments, the composition comprises the ionic liquid in a concentration of about 20% to 40% and a gel base in a concentration of about 60% to 80%. In some embodiments, the composition comprises the ionic liquid in a concentration of 20% and a gel base in a concentration of 80%. In some embodiments, the composition comprises the ionic liquid in a concentration of about 20% to 40%, propylene glycol in a concentration of 20-50%, glycerin in a concentration of 10-20%, ethanol in a concentration of about 10-20%, and hydroxyl propyl cellulose in a concentration of less than 5%.

In some embodiments, the composition comprises propylene glycol. In some embodiments, the concentration of propylene glycol in the composition is about 1%, 5%, 10%, 20%, 30%, 40%, or 50%. In some embodiments, the concentration of propylene glycol in the composition is in a range of 1% to 40%, 5% to 20%, or 10% to 15%.

In some embodiments, the composition comprises hydroxyethyl cellulose. In some embodiments, the concentration of hydroxyethyl cellulose in the composition is about 0.1%, 0.5%, 1.0%, 2.0%, 3.0%, 4.0%, or 5.0%. In some embodiments, the concentration of hydroxyethyl cellulose in the composition is in a range of 0.5% to 5.0%, 0.75% to 2.0%, or 1.0% to 1.5%.

In some embodiments, the composition comprises a fragrance agent. In some embodiments, the fragrance agent comprises or is derived from essential oils, absolutes, resinoids, resins, concretes, or synthetic perfume components such as hydrocarbons, alcohols, aldehydes, ketones, ethers, acids, acetals, ketals and nitriles, including saturated and unsaturated compounds, aliphatic, carbocyclic and heterocyclic compounds, or precursors of any of the above. Exemplary fragrant agents include, but are not limited to, eucalyptus (Eucalyptus globulus or Eucalyptus citriadora), pine needles (Picca excelsa), Ho-leaves (Cinnamomum camphora hosch), peppermint (Mentha piperita), neem tree (Azadirachta excelsa), bay leaves (Laurus nobilis), litsea (Litsea cubeba), citronella (Cymbopogon nardus), elemi (Canarium luzonicum), petitgrain citronniers lemon (Citrus limonum), grapefruit (Citrus paradisi), fir tree (Abies alba pectinata), lavender (Lavandula officinalis), bergamotte (Citrus aurantium bergamia), and rosemary (Rosmarinus officinalis). In some embodiments, the fragrance agent is derived from a citrus fruit including but not limited to, oranges, lemons, grapefruit, and limes. In some embodiments, the fragrance agent is an acid or terpene derived from a citrus fruit. In some embodiments, the fragrance agent is citric acid or a citric acid derivative. In some embodiments, the fragrance agent is limonene.

In some embodiments, the composition comprises D-limonene. In some embodiments, the concentration of D-limonene in the composition is about 0.1%, 0.5%, 1.0%, 2.0%, 3.0%, 4.0%, or 5.0%. In some embodiments, the concentration of D-limonene in the composition is in a range of 0.5% to 5.0%, 0.75% to 2.0%, or 1.0% to 1.5%.

In some embodiments, the composition comprises the ionic liquid in a concentration of about 5% to 40% and a gel base comprising the pharmaceutically acceptable solvent in a concentration of about 60% to 95%. In some embodiments, the composition comprises the ionic liquid in a concentration of about 5% to 40%, and a gel base in a concentration of about 60% to 95%, wherein the gel base comprises diisopropyl adipate, propylene glycol, and a poloxamer. In some embodiments, the poloxamer is a Pluronic®.

In some embodiments, the composition comprises the ionic liquid in a concentration of about 1% to 50%, and the pharmaceutically acceptable solvent in a concentration of about 50% to 99%. In some embodiments, the composition comprises the ionic liquid in a concentration of about 1% to 50%, and water in a concentration of about 50% to 99%. In some embodiments, the water is deionized water or Milli-Q® water.

In some embodiments, the composition comprises the ionic liquid in a concentration of about 1% to 50%, a pharmaceutically acceptable solvent in a concentration of about 1% to 50%, and a gelling agent in a concentration of about 1 to 5%. In some embodiments, the composition comprises the ionic liquid in a concentration of about 1% to 50%, water in a concentration of about 1% to 50%, and HPC in a concentration of about 1 to 5%.

In some embodiments, the pharmaceutically acceptable solvent is diisopropyl adipate. In some embodiments, the composition comprises diisopropyl adipate in a concentration of about 20%. In some embodiments, the composition comprises the ionic liquid in a concentration of about 1% to 40%, and diisopropyl adipate in a concentration of about 60% to about 99%.

In some embodiments, the composition comprises a gel base in a concentration of about 50% to 90% of the composition. In some embodiments, the composition comprises a gel base in a concentration of about 50%, 60%, 70%, 80%, or 90% of the composition.

In some embodiments, preparing an ionic liquid comprising a choline cation and a fatty acid anion comprises: (a) mixing choline and a fatty acid in a solvent at room temperature in a predetermined ratio; and (b) removing the solvent in vacuo. In some embodiments, the fatty acid is geranic acid. In some embodiments, the solvent is water. In a particular embodiment, the water is deionized water. In some embodiments, removing the solvent comprises rotary evaporation. In some embodiments, removing the solvent comprises heating the ionic liquid, applying a vacuum to the ionic liquid, or a combination thereof. In some embodiments, preparing the ionic liquid further comprises drying the ionic liquid. In some embodiments, heating the ionic liquid comprises heating the ionic liquid to 60° C. In some embodiments, the heating is done for at least 10 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours. In some embodiments, the vacuum is applied at −100 kPa. In some embodiments, the vacuum is applied for at least 10 minutes, 20 minutes, 30 minutes, 1 hours, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours.

In some embodiments, the ionic liquid has had the solvent used in the ionic liquid preparation process removed. In some embodiments, the ionic liquid does not comprise water.

In some embodiments, choline is choline bicarbonate. In some embodiments, the choline is choline in an 80% wt solution of choline bicarbonate. In some embodiment, the predetermined ratio is a ratio of 1:1, 1:2, 1:3, or 1:4 of a choline cation:fatty acid anion. In one embodiment, the ratio is a molar ratio. In another embodiment, the ratio is ratio by weight.

In some embodiments, isolating the composition further comprises purifying the ionic liquid. In some embodiments, purifying the ionic liquid comprises using conventional techniques, including, but not limited to, filtration, distillation, crystallization, and chromatography. In some embodiments, preparing the ionic liquid further comprises isolating the purified ionic liquid

### Methods of Treating Skin Conditions

Disclosed herein, in certain embodiments, are methods for treating a skin condition in an individual in need thereof comprising administering to a skin of the individual a composition comprising: (a) an ionic liquid comprising a choline cation and a fatty acid anion; and (b) a pharmaceutically acceptable solvent. In some embodiments, the fatty acid anion is a geranic acid anion. In some embodiments, the individual is a mammal. In some embodiments, the mammal is a human.

In some embodiments, the skin condition is associated with infection. In some embodiments, the skin condition is associated with inflammation. In some embodiments, the skin condition is associated with inflammation and infection. In some embodiments, skin conditions associated with infection show symptoms of lesions, papules, pustules, or a combination thereof. In some embodiments, skin conditions associated with inflammation show symptoms of rashes, redness (erythema), persistent red veins, or a combination thereof.

In some embodiments, the skin condition is caused by a mite, bacteria, virus, yeast, or fungus. In some embodiments, the skin condition is caused by a pathogen. In some embodiments, the pathogen includes, but is not limited to, dermatophytes, non-dermatophyte molds, and yeasts. In some embodiments, the yeast is a Candida species. In some embodiments, the dermatophyte is a Trichophyton species. In some embodiments, the dermatophyte is Trichophyton rubrum. In some embodiments, the skin condition is caused by a virus. In some embodiments, the virus is molluscum contagiosum virus (MCV). In some embodiments, the MCV is MCV-1, MCV-2, MCV-3, or MCV-4. In some embodiments, treatment of the skin condition with the composition does not induce development of resistance in the mite, bacteria, virus, yeast, or fungus. In some embodiments, the mite is a Demodex mite. In some embodiments, the Demodex mite is Demodex folliculorum or Demodex brevis. In some embodiments, the bacteria is Bacillus oleronius or Staphylococcus epidermidis. In some embodiments, the bacteria is associated with the Demodex mite. In some embodiments, the rosacea is associated with proliferation of Demodex mites.

In some embodiments, the skin condition is rosacea. In some embodiments, the skin condition is impetigo, cold sore, wart, molluscum contagiosum, onychomycosis, rosacea, or a combination thereof. In some embodiments, the onychomycosis is distal and lateral subungual onychomycosis (DLSO). In some embodiments, the onychomycosis is superficial white onychomycosis (SWO). In some embodiments, the onychomycosis is proximal subungual onychomycosis (PSO). In some embodiments, the onychomycosis is candidial onychomycosis. In some embodiments, the onychomycosis is total dystrophic onychomycosis. In some embodiments, the skin condition is a skin condition caused by an overpopulation of Demodex mites, such as demodicosis.

In some embodiments, the skin condition causes erythema, inflammation, lesions, or a combination thereof on the skin of the individual. In some embodiments, the skin condition causes papule, nodules, redness, inflammation, and a combination thereof on the skin. In some embodiments, the condition causes a nail plate having a thickened, yellow, or cloudy appearance, nails that are rough, or nails that separate from the nail bed. In some embodiments, a therapeutically effective amount of the composition is administered to the skin of the individual. In some embodiments, the composition is administered to an area of skin affected with the skin condition. In some embodiments, therapeutically effective amounts are determined by routine experimentation, including but not limited to a dose escalation clinical trial. In some embodiments, administration of the composition to the skin of the individual results in a reduction of erythema of the skin of the individual. In some embodiments, administration of the composition to the skin of the individual results in a reduction of inflammation of the skin of the individual. In some embodiments, inflammation is reduced by down regulating a cytokine. In some embodiment, the cytokine is a tumor necrosis factor alpha (TNFα), an interleukin, and a combination thereof. In some embodiments, administration of the composition to the skin of the individual results in a reduction of lesions on the skin of the individual. In some embodiments, administration of the composition to the skin of the individual results in a reduction of papule, nodules, redness, inflammation, or a combination thereof on the skin. In some embodiments, administration of the composition to the individual results in a reduction of nail plate having a thickened, yellow, or cloudy appearance, nails that are rough, or nails that separate from the nail bed.

In some embodiments, the composition is administered prophylactically to an individual susceptible or otherwise at risk of the skin condition.

In some embodiments, the amount of the composition administered to the individual and the length of treatment depends on the attributes of the individual including, but not limited to, state of health, weight, severity of the condition, previous therapy, and judgement of the treating physician. In some embodiments, the amount of the composition administered to the individual is determined by routine experimentation (e.g., a dose escalation clinical trial).

In some embodiments, the composition is applied to the skin of the individual once a day. In some embodiments, the composition is applied to the skin of the individual 1, 2, 3, 4, or 5 times a day. In some embodiments, the composition is applied to the skin of the individual 2 times a day. In some embodiments, the composition is applied to the skin of the individual 2 times a day, e.g., morning and evening. In some embodiments, the composition is applied to the skin of the individual every day, every other day, every three days, twice a week, once a week, or once a month. In some embodiments, the composition is applied to the skin of the individual once. In some embodiments, the composition is applied to the skin of the individual for a period of time of 1 week, 2 weeks, 3 weeks, 1 month, 2 months, or 3 months. In some embodiments, the composition is applied to the skin until the symptoms of the skin condition associated with infection are eliminated. In some embodiments, the composition is applied to the skin until the symptoms of the skin condition associated with inflammation are eliminated. In some embodiments, the composition is applied to the skin until the symptoms of the skin condition associated with infection are reduced. In some embodiments, the composition is applied to the skin until the symptoms of the skin condition associated with inflammation are reduced.

In some embodiments, compositions as described herein improve the symptoms of rosacea. In some embodiments, compositions as described herein decrease the number of inflammatory lesions. In some embodiments, compositions as described herein decrease the number of inflammatory lesions by 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein decrease the number of inflammatory lesions by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. In some embodiments, compositions as described herein decrease the redness of the skin. In some embodiments, compositions as described herein decrease the redness of the skin by 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein decrease the redness of the skin by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. In some embodiments, compositions as described herein improve skin complexion. In some embodiments, improved skin complexion comprises a reduction in redness, bumps, blemishes, or a combination thereof. In some embodiments, compositions as described herein improve skin complexion by 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein improve skin complexion by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×.

In some embodiments, compositions as described herein improve the symptoms of rosacea by a certain time. In some embodiments, compositions as described herein decrease the number of inflammatory lesions following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease the number of inflammatory lesions by at least or about 10%, 15%, 20%, 25%, 30%, 35%, 40%, 45%, 50%, 55%, 60%, 65%, 70%, 75%, 80%, 85%, 90%, 95%, or more than 95% following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease the redness of the skin following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease the redness of the skin by at least or about 10%, 15%, 20%, 25%, 30%, 35%, 40%, 45%, 50%, 55%, 60%, 65%, 70%, 75%, 80%, 85%, 90%, 95%, or more than 95% following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease redness, bumps, blemishes, or a combination thereof following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease redness, bumps, blemishes, or a combination thereof by at least or about 10%, 15%, 20%, 25%, 30%, 35%, 40%, 45%, 50%, 55%, 60%, 65%, 70%, 75%, 80%, 85%, 90%, 95%, or more than 95% following at least or about 1 day, 2 days, 3 days, 4 days, 5 days, 6 days, 1 week, 2 weeks, 3 weeks, 1 month, 2 months, 3 months, or more than 3 months of administration.

Methods for determining improvements in the symptoms of rosacea, in some embodiments, comprise the Investigator's Global Assessment, the Investigator's Global Assessment of Redness, the Subject Global Assessment, or a combination thereof.

In some embodiments, administration of the composition to the skin of the individual results in a reduction in a viral infection. In some embodiments, compositions as described herein improve the symptoms of molluscum contagiosum (molluscum). In some embodiments, compositions as described herein reduce a number of papules or nodules by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein decrease inflammation of the skin by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein decrease the redness of the skin by at least or about 10%, 15%, 20%, 25%, 30%, 35%, 40%, 45%, 50%, 55%, 60%, 65%, 70%, 75%, 80%, 85%, 90%, 95%, or more than 95%.

In some embodiments, compositions as described herein improve the symptoms of a viral infection by a certain time. In some embodiments, compositions as described herein improve the symptoms of molluscum contagiosum (molluscum) by a certain time. In some embodiments, compositions as described herein reduce a number of papules or nodules following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease inflammation of the skin by at least or about 10%, 15%, 20%, 25%, 30%, 35%, 40%, 45%, 50%, 55%, 60%, 65%, 70%, 75%, 80%, 85%, 90%, 95%, or more than 95% following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease the redness of the skin following at least or about 1 day, 2 days, 3 days, 4 days, 5 days, 6 days, 1 week, 2 weeks, 3 weeks, 1 month, 2 months, 3 months, or more than 3 months of administration.

In some embodiments, administration of the composition of the individual results in a reduction of fungal or pathogen infection. In some embodiments, compositions as described herein improve the symptoms of a fungal or pathogen infection by a certain time. In some embodiments, compositions as described herein improve the symptoms of onychomycosis by a certain time. In some embodiments, the composition is administered to the epidermis and dermis to treat onychomycosis. In some embodiments, the composition is administered to a nail substrate to treat onychomycosis. In some embodiments, the composition penetrates to the epidermis and dermis. In some embodiments, the composition penetrates to a nail substrate. In some embodiments, the nail substrate comprises the nail plate, the nail matrix, the nail bed, or a combination thereof. In some embodiments, the composition penetrates to a nail substrate and accumulates at a dose of at least or about 0.01, 0.05, 0.1, 0.5, 1, 5, 10, 25, 50, 75, 100, 125, 150, 175, 200, 225, 250, 275, 300, 325, 350, 375, 400, 425, 450, 475, 500, 525, 550, 575, 500, 525, 550, 575, 600, 625, 650, 675, 700, 825, 850, 875, 900, 925, 950, 1000, or more than 100 microgram per centimeter squared (μg/cm²). In some embodiments, the composition penetrates to a nail substrate and accumulates at a dose of at least or about 0.01, 0.05, 0.1, 0.5, 1, 5, 10, 25, 50, 75, 100, 125, 150, 175, 200, 225, 250, 275, 300, 325, 350, 375, 400, 425, 450, 475, 500, 525, 550, 575, 500, 525, 550, 575, 600, 625, 650, 675, 700, 825, 850, 875, 900, 925, 950, 1000, or more than 1000 microgram per centimeter squared (μg/cm²). In some embodiments, the composition penetrates to a nail substrate and accumulates at a dose of at least or about 0.01, 0.05, 0.1, 0.5, 1, 5, 10, 25, 50, 75, 100, 125, 150, 175, 200, 225, 250, 275, 300, 325, 350, 375, 400, 425, 450, 475, 500, 525, 550, 575, 500, 525, 550, 575, 600, 625, 650, 675, 700, 825, 850, 875, 900, 925, 950, 1000, or more than 1000 microgram per centimeter squared (μg/cm²) after 1, 2, 3, 4, 5, 6, 7, 8, 12, 16, 18, 20, 24, 28, 32, 36, 40, or more than 40 days after administration. In some embodiments, administration of the composition to the individual results in a reduction of symptoms associated with onychomycosis. In some embodiments, administration of the composition to the individual results in an improvement of symptoms associated with onychomycosis. For example, symptoms include a nail plate having a thickened, yellow, or cloudy appearance, nails that are rough, or nails that separate from the nail bed. In some embodiments, compositions as described herein improve symptoms associated with onychomycosis by 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein improve symptoms associated with onychomycosis by 10%, 15%, 20%, 25%, 30%, 35%, 40%, 45%, 50%, 55%, 60%, 65%, 70%, 75%, 80%, 85%, 90%, 95%, or more than 95% following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein improve symptoms associated with onychomycosis by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. In some embodiments, compositions as described herein improve symptoms associated with onychomycosis by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10× following at least or about 1 day, 2 days, 3 days, 4 days, 5 days, 6 days, 1 week, 2 weeks, 3 weeks, 1 month, 2 months, 3 months, or more than 3 months of administration.

In some embodiments, administration of the composition is temporarily reduced or temporarily suspended for a certain length of time (i.e., a “drug holiday”). In some embodiments, the length of the drug holiday is between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. In some embodiments, the dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

### Certain Terminology

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for rosacea, if, after receiving a therapeutic amount of a composition according to the methods of the present disclosure, the subject shows observable and/or measurable reduction in or absence of one or more of the following: reduction in the erythema; reduction in the appearance of red veins; papules, and pustules.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### NUMBERED EMBODIMENTS

The disclosure is further elucidated by reference to the numbered embodiments herein. Numbered embodiment 1 comprises a method for treating an inflammatory or infectious skin disease or condition in an individual in need thereof, comprising administering to a skin of the individual a composition comprising: (a) an ionic liquid comprising a choline cation and geranic acid anion; and (b) a pharmaceutically acceptable solvent. Numbered embodiment 2 comprises the method of numbered embodiment 1, wherein the inflammatory or infectious skin disease or condition is rosacea, molluscum contagiosum, or onychomycosis. Numbered embodiment 3 comprises the method of numbered embodiments 1-2, wherein the inflammatory or infectious skin disease or condition is rosacea. Numbered embodiment 4 comprises the method of numbered embodiments 1-3, the skin disease or condition is an inflammatory skin disease or condition. Numbered embodiment 5 comprises the method of numbered embodiments 1-4, wherein the pharmaceutically acceptable solvent is selected from the group consisting of: water, ethanol, diisopropyl adipate, polyethylene glycol (PEG), glycerin, propylene glycol, and a combination thereof. Numbered embodiment 6 comprises the method of numbered embodiments 1-5, wherein the composition further comprises a gelling agent. Numbered embodiment 7 comprises the method of numbered embodiments 1-6, wherein the gelling agent is selected from the group consisting of: hydroxyethyl cellulose (HEC), hydroxypropyl cellulose (HPC), hydroxypropylmethyl cellulose (HPMC), and a combination thereof. Numbered embodiment 8 comprises the method of numbered embodiments 1-7, wherein the ionic liquid comprises the choline cation and geranic acid anion in a molar ratio in a range of 1:1 to 1:4 of choline cation to geranic acid anion. Numbered embodiment 9 comprises the method of numbered embodiments 1-8, wherein the ionic liquid comprises the choline cation and geranic acid anion in a molar ratio of 1:1, 1:2, 1:3, or 1:4 of choline cation to geranic acid anion. Numbered embodiment 10 comprises the method of numbered embodiments 1-9, wherein the composition provides an increased antimicrobial action compared to an antimicrobial action of choline or an antimicrobial action of geranic acid. Numbered embodiment 11 comprises the method of numbered embodiments 1-10, wherein the increased antimicrobial action is a 10 fold less concentration of the composition required for complete killing of a microbe relative to a concentration of choline or a concentration of geranic acid required for complete killing of the microbe. Numbered embodiment 12 comprises the method of numbered embodiments 1-11, wherein the composition provides an increased skin permeation relative to a skin permeation of choline or a skin permeation of geranic acid. Numbered embodiment 13 comprises the method of numbered embodiments 1-12, wherein the composition provides an increased conductivity relative to a conductivity of geranic acid and a decreased conductivity relative to a conductivity of choline. Numbered embodiment 14 comprises the method of numbered embodiments 1-13, wherein the ionic liquid is present at a concentration of about 0.1% to 99% of the composition, and the pharmaceutically acceptable solvent comprises a concentration of about 1% to about 99.9% of the composition. Numbered embodiment 15 comprises the method of numbered embodiments 1-14, wherein the composition is formulated for transdermal administration. Numbered embodiment 16 comprises the method of numbered embodiments 1-15, wherein the composition further comprises an additional therapeutic agent selected from the group consisting of: a small molecule drug, an antimicrobial agent, a protein, a peptide, an antibody, a nucleic acid, a chemotherapy agent, and a combination thereof. Numbered embodiment 17 comprises the method of numbered embodiments 1-16, wherein the composition is formulated as a gel, lotion, cream, ointment, solution, or a patch. Numbered embodiment 18 comprises the method of numbered embodiments 1-17, wherein erythema of the skin of the individual is reduced. Numbered embodiment 19 comprises the method of numbered embodiments 1-18, wherein redness of the skin is reduced. Numbered embodiment 20 comprises the method of numbered embodiments 1-19, wherein inflammation of the skin of the individual is reduced. Numbered embodiment 21 comprises the method of numbered embodiments 1-20, wherein a number of lesions on the skin is reduced. Numbered embodiment 22 comprises the method of numbered embodiments 1-21, wherein lesions on the skin of the individual are reduced. Numbered embodiment 23 comprises a method for treating rosacea in an individual in need thereof, comprising administering to a skin of the individual a composition comprising: (a) an ionic liquid comprising a choline cation and a geranic acid anion; and (b) a pharmaceutically acceptable solvent selected from the group consisting of: diisopropyl adipate, polyethylene glycol (PEG), glycerin, propylene glycol, and a combination thereof. Numbered embodiment 24 comprises a method for treating rosacea in an individual in need thereof, comprising administering to a skin of the individual a composition comprising: (a) an ionic liquid comprising a choline cation and a geranic acid anion; (b) a pharmaceutically acceptable solvent selected from the group consisting of: water, ethanol, diisopropyl adipate, polyethylene glycol (PEG), glycerin, propylene glycol, and a combination thereof; and (c) a gelling agent. Numbered embodiment 25 comprises the method of numbered embodiments 1-24, wherein the rosacea is caused by a mite, bacteria, or a combination thereof. Numbered embodiment 26 comprises the method of numbered embodiments 1-25, wherein the composition does not induce development of resistance in the mite or the bacteria. Numbered embodiment 27 comprises the method of numbered embodiments 1-26, wherein erythema of the skin of the individual is reduced. Numbered embodiment 28 comprises the method of numbered embodiments 1-27, wherein redness of the skin is reduced. Numbered embodiment 29 comprises the method of numbered embodiments 1-28, wherein inflammation of the skin of the individual is reduced. Numbered embodiment 30 comprises the method of numbered embodiments 1-29, wherein a number of lesions on the skin is reduced. Numbered embodiment 31 comprises the method of numbered embodiments 1-30, wherein lesions on the skin of the individual are reduced. Numbered embodiment 32 comprises the method of numbered embodiments 1-31, wherein the ionic liquid comprises the choline cation and geranic acid anion in a range of 1:1 to 1:4 of choline cation to geranic acid. Numbered embodiment 33 comprises the method of numbered embodiments 1-32, wherein the ionic liquid comprises the choline cation and geranic acid anion in a molar ratio of 1:1, 1:2, 1:3, or 1:4 of choline cation to geranic acid anion. Numbered embodiment 34 comprises the method of numbered embodiments 1-33, wherein the composition provides an increased antimicrobial action compared to an antimicrobial action of choline or an antimicrobial action of geranic acid. Numbered embodiment 35 comprises the method of numbered embodiments 1-34, wherein the increased antimicrobial action is a 10 fold less concentration of the composition required for complete killing of a microbe relative to a concentration of choline or a concentration of geranic acid required for complete killing of the microbe. Numbered embodiment 36 comprises the method of numbered embodiments 1-35, wherein the composition provides an increased skin permeation relative to a skin permeation of choline or a skin permeation of geranic acid. Numbered embodiment 37 comprises the method of numbered embodiments 1-36, wherein the composition provides an increased conductivity relative to a conductivity of geranic acid and a decreased conductivity relative to a conductivity of choline. Numbered embodiment 38 comprises the method of numbered embodiments 1-37, wherein the ionic liquid is present a concentration of about 0.1% to 99% of the composition, and the pharmaceutically acceptable solvent comprises a concentration of about 1% to about 99.9% of the composition. Numbered embodiment 39 comprises the method of numbered embodiments 1-38, wherein the composition is formulated for transdermal administration. Numbered embodiment 40 comprises the method of numbered embodiments 1-39, further comprising an additional therapeutic agent selected from the group consisting of: a small molecule drug, an antimicrobial agent, a protein, a peptide, an antibody, a nucleic acid, a chemotherapy agent, and a combination thereof. Numbered embodiment 41 comprises the method of numbered embodiments 1-40, wherein the composition is formulated as a gel, lotion, cream, ointment, solution, or a patch. Numbered embodiment 42 comprises the method of numbered embodiments 1-41, wherein the gelling agent is selected from the group consisting of: hydroxyethyl cellulose (HEC), hydroxypropyl cellulose (HPC), hydroxypropylmethyl cellulose (HPMC), and a combination thereof. Numbered embodiment 43 comprises the method of numbered embodiments 1-42, further comprising a fragrance agent. Numbered embodiment 44 comprises the method of numbered embodiments 1-43, wherein the fragrance agent is an acid or a terpene of a citrus fruit. Numbered embodiment 45 comprises the method of numbered embodiments 1-44, wherein the citrus fruit is an orange, a grapefruit, a lime, or a lemon. Numbered embodiment 46 comprises the method of numbered embodiments 1-45, wherein the terpene is D-limonene. Numbered embodiment 47 comprises the method of numbered embodiments 1-46, wherein the acid is citric acid or a derivative thereof. Numbered embodiment 48 comprises a composition comprising: (a) an ionic liquid comprising a choline cation and a geranic acid anion; and (b) a pharmaceutically acceptable solvent selected from the group consisting of: diisopropyl adipate, polyethylene glycol (PEG), glycerin, propylene glycol, and a combination thereof. Numbered embodiment 49 comprises a composition comprising (a) an ionic liquid comprising a choline cation and a geranic acid anion; (b) a pharmaceutically acceptable solvent selected from the group consisting of: water, ethanol, diisopropyl adipate, polyethylene glycol (PEG), glycerin, propylene glycol, and a combination thereof; and (c) a gelling agent. Numbered embodiment 50 comprises a composition of numbered embodiments 1-49, wherein the ionic liquid comprises the choline cation and geranic acid anion in a molar ratio in a range of 1:1 to 1:4 of choline cation to geranic acid anion. Numbered embodiment 51 comprises a composition of numbered embodiments 1-50, wherein the ionic liquid comprises the choline cation and geranic acid anion in a molar ratio of 1:1, 1:2, 1:3, or 1:4 of choline cation to geranic acid anion. Numbered embodiment 52 comprises a composition of numbered embodiments 1-51, wherein the composition provides an increased antimicrobial action compared to an antimicrobial action of choline or an antimicrobial action of geranic acid. Numbered embodiment 53 comprises a composition of numbered embodiments 1-52, wherein the increased antimicrobial action is a 10 fold less concentration of the composition required for complete killing of a microbe relative to a concentration of choline or a concentration of geranic acid required for complete killing of the microbe. Numbered embodiment 54 comprises a composition of numbered embodiments 1-53, wherein the composition provides an increased skin permeation relative to a skin permeation of choline or a skin permeation of geranic acid. Numbered embodiment 55 comprises a composition of numbered embodiments 1-54, wherein the composition provides an increased conductivity relative to a conductivity of geranic acid and a decreased conductivity relative to a conductivity of choline. Numbered embodiment 56 comprises a composition of numbered embodiments 1-55, wherein the ionic liquid comprises a concentration of about 0.1% to 99% of the composition, and the pharmaceutically acceptable solvent comprises a concentration of about 1% to about 99.9% of the concentration. Numbered embodiment 57 comprises a composition of numbered embodiments 1-56, wherein the composition is formulated for transdermal administration. Numbered embodiment 58 comprises a composition of numbered embodiments 1-57, further comprising an additional therapeutic agent selected from the group consisting of: a small molecule drug, an antimicrobial agent, a protein, a peptide, an antibody, a nucleic acid, a chemotherapy agent, and a combination thereof. Numbered embodiment 59 comprises a composition of numbered embodiments 1-58, wherein the composition is formulated as a gel, lotion, cream, ointment, solution, or a patch. Numbered embodiment 60 comprises a composition of numbered embodiments 1-59, wherein the gelling agent is selected from the group consisting of: hydroxyethyl cellulose (HEC), hydroxypropyl cellulose (HPC), hydroxypropylmethyl cellulose (HPMC), and a combination thereof. Numbered embodiment 61 comprises a composition of numbered embodiments 1-60, further comprising a fragrance agent. Numbered embodiment 62 comprises a composition of numbered embodiments 1-61, wherein the fragrance agent is an acid or a terpene of a citrus fruit. Numbered embodiment 63 comprises a composition of numbered embodiments 1-62, wherein the citrus fruit is an orange, a grapefruit, a lime, or a lemon. Numbered embodiment 64 comprises a composition of numbered embodiments 1-63, wherein the terpene is D-limonene. Numbered embodiment 65 comprises a composition of numbered embodiments 1-64, wherein the acid is citric acid or a derivative thereof. Numbered embodiment 66 comprises a composition comprising 20% to 60% of an ionic liquid comprising a choline cation and a geranic acid anion, 5% to 20% propylene glycol, and a remaining balance of water. Numbered embodiment 67 comprises a composition of numbered embodiments 1-66 comprising 30% to 50% of the ionic liquid. Numbered embodiment 68 comprises a composition of numbered embodiments 1-67, wherein a molar ratio of the choline cation and geranic acid anion is 1:2. Numbered embodiment 69 comprises a composition of numbered embodiments 1-68 comprising 10% to 15% propylene glycol. Numbered embodiment 70 comprises a composition of numbered embodiments 1-69, wherein the composition further comprises 0.5% to 5% hydroxyethyl cellulose. Numbered embodiment 71 comprises a composition of numbered embodiments 1-70, wherein the composition further comprises 0.5% to 5% D-limonene. Numbered embodiment 72 comprises a composition of numbered embodiments 1-71, wherein the composition is formulated as a gel. Numbered embodiment 73 comprises a composition of numbered embodiments 1-72, wherein the composition is formulated for topical administration. Numbered embodiment 74 comprises a composition of numbered embodiments 1-73, wherein the composition is formulated for twice daily administration.

### EXAMPLES

### Example 1: Preparation of a Composition Comprising Choline and Geranic Acid in a 1:1 Molar Ratio (Composition B)

The purified GMP Penta Geranic acid (311.0 g, 1.848 mol) was placed in a 2 L round bottomed flask. The flask was placed in a water bath at 20° C. and stirred. Then choline bicarbonate (381.7 g, 1.848 mol) 80% solution in water (Sigma, C7519, 209 ml) was added slowly (drop-wise) with an addition funnel, total addition time was 120 min. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −100 kPa to remove the resulting water. After no more water evaporation was observed by condensation on the dry ice trap of the rotavap, the flask was further heated at 60° C. and −100 kPa for 36 additional hrs to dry the final product. 475 g of product (94.7% yield) was obtained. HPLC analysis shows 97.9% purity.

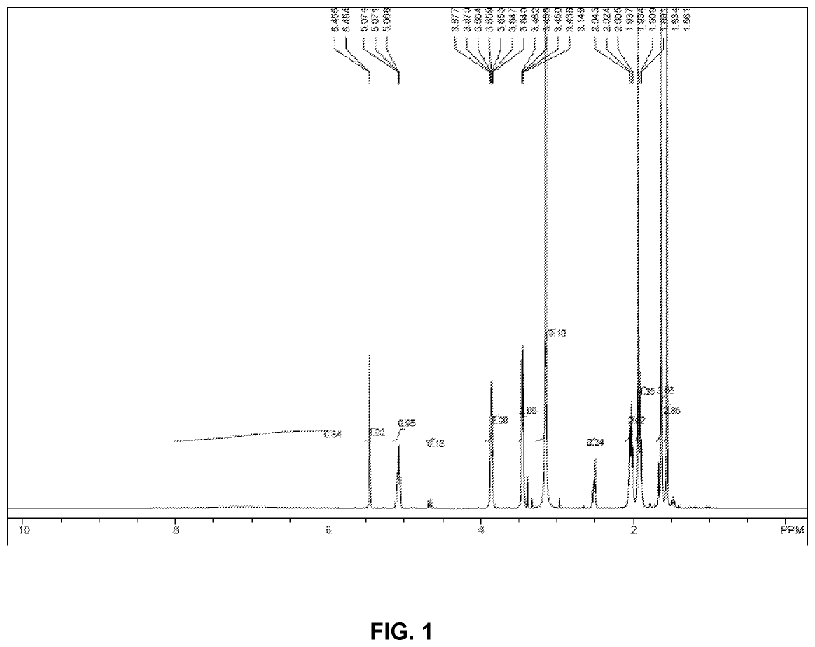

¹H NMR spectra is depicted in FIG. 1.

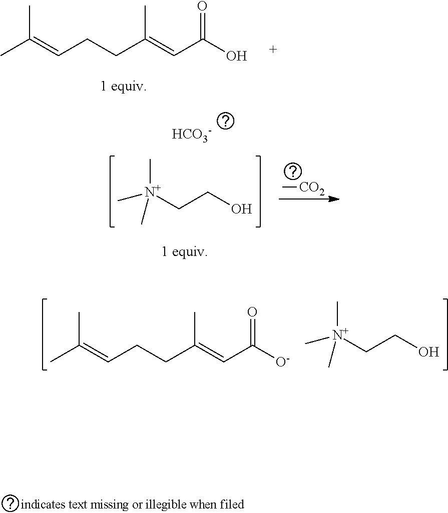

### Example 2: Preparation of a Composition Containing Choline and Geranic Acid in a 1:2 Molar Ratio (Composition A)

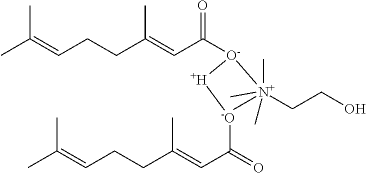

To two equivalents (9.88 g., 0.059 moles) of neat geranic acid, recrystallized 5× at −70° C. from 70% geranic acid/30% acetone, in a 500 mL round bottom flask was added one equivalent of choline bicarbonate (80 wt % solution, 6.06 g, 0.029 mol). The mixture was stirred at room temperature until no more CO₂ evolved. Solvent was removed by rotary evaporation at 60° C. for 20 min, and the product was dried in a vacuum oven for 48 h at 60° C.

Physical characterization at 25° C.: solubility in water=0.5 M; density=0.990 g/mL; conductivity=0.0431 mS/cm; viscosity=1345 cP.

### Example 3: Alternate Preparation of a Composition Containing Choline and Geranic Acid in a 1:2 Molar Ratio (Composition A)

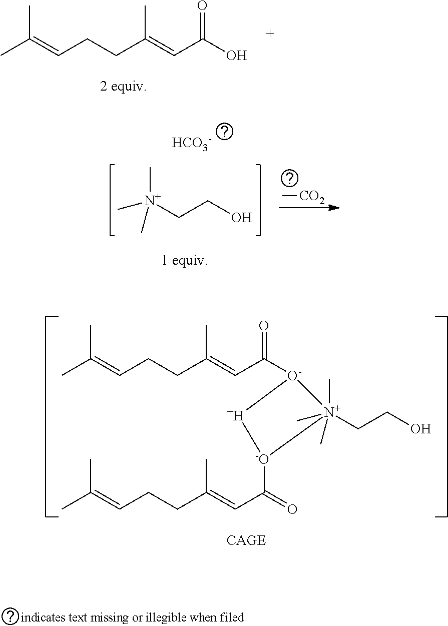

The purified GMP Penta Geranic acid (155 g, 0.921 mol) was placed in a 1 L round bottomed flask. The flask was placed in a water bath at 20° C. and stirred. Then choline bicarbonate (95.1 g, 0.460) 80% solution in water (Sigma, C7519, Lot #: 059K1526V, 209 ml) was added slowly (drop-wise) with an addition funnel, total addition time was 35 min. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −100 kPa to remove the resulting water. After no more water evaporation was observed by condensation on the dry ice trap of the rotavap, the flask was further heated at 60° C. and −100 kPa for 36 additional hrs to dry the final product. 197 g of Cage (96% yield) was obtained. 1H-NMR spectrum looks similar to the one of CB-0001. HPLC analysis shows 95.1% purity.

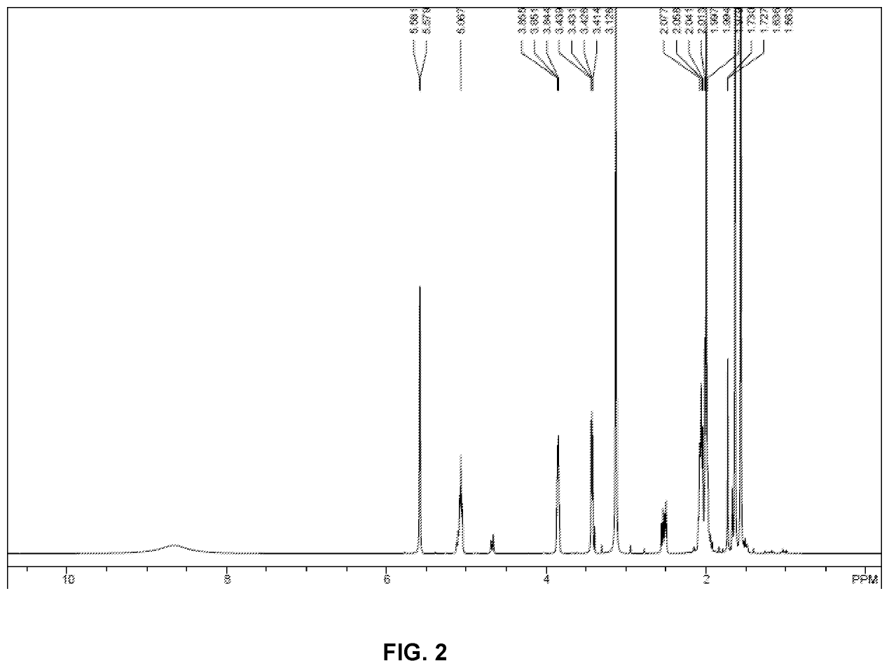

¹H NMR spectra is depicted in FIG. 2.

### Example 4: Preparation of a Composition Containing Choline and Geranic Acid in a 1:3 Molar Ratio

To three equivalents (14.56 g., 0.087 moles) of neat geranic acid, recrystallized 5× at −70° C. from 70% geranic acid/30% acetone, in a 1000 mL round bottom flask is added one equivalent of choline bicarbonate (80 wt % solution, 6.06 g, 0.029 mol). The mixture is stirred at room temperature until no more CO₂ evolved. Solvent is removed by rotary evaporation at 60° C. for 20 min, and the product is dried in a vacuum oven for 48 h at 60° C.

### Example 5: Preparation of a Composition Containing Choline and Geranic Acid in a 1:4 Molar Ratio

To four equivalents (19.76 g., 0.118 moles) of neat geranic acid, recrystallized 5× at −70° C. from 70% geranic acid/30% acetone, in a 800 mL round bottom flask is added one equivalent of choline bicarbonate (80 wt % solution, 6.06 g, 0.029 mol). The mixture is stirred at room temperature until no more CO₂ evolved. Solvent is removed by rotary evaporation at 60° C. for 20 min, and the product is dried in a vacuum oven for 48 h at 60° C.

### Example 6: Conductivity of Choline and Geranic Acid in Solvents

Conductivity in mixtures of a solvent in combination with choline and geranic acid were done separately. Geranic acid contributed effectively zero to the overall conductivity. Measuring the choline bicarbonate alone gave values higher than those with samples made with Composition A. A sample of geranic acid and choline (bicarbonate) was made separately before adding to the solvent of interest. The choline solution was not miscible with diisopropyl adipate, while Composition A was miscible with diisopropyl adipate. Propylene glycol and ethanol diluted out the conductivity of the choline differently.

Propylene glycol: Samples were made by first starting with 5 grams of each raw material; geranic acid first then repeated with choline (bicarbonate). In summary 1-3 grams of propylene glycol was added to the raw material being tested and mixed. A visual observation was made along with a conductivity measurement.

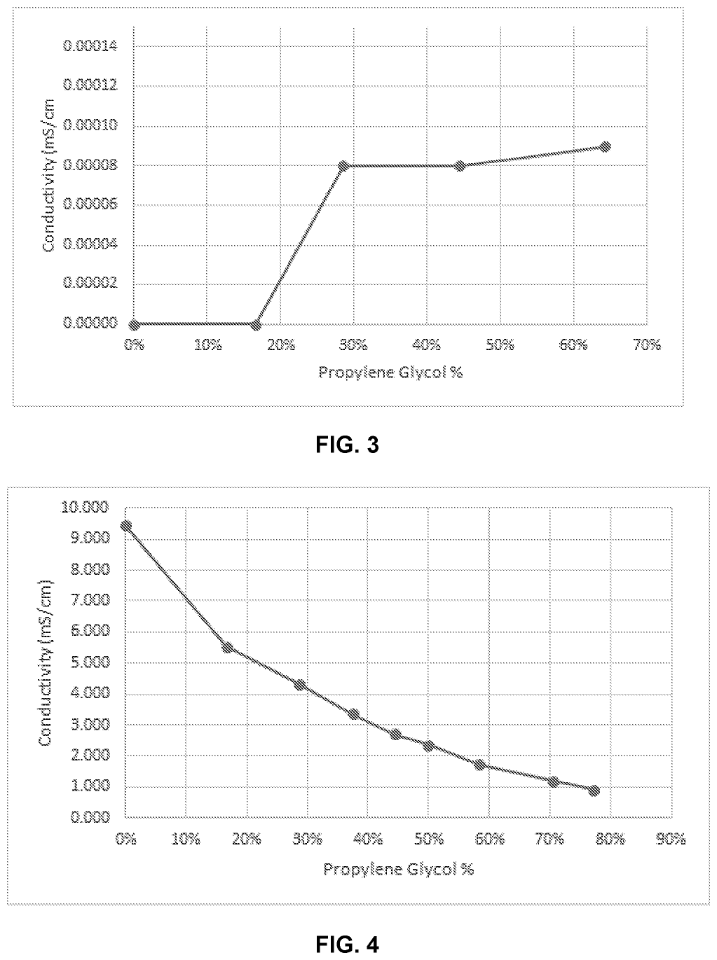

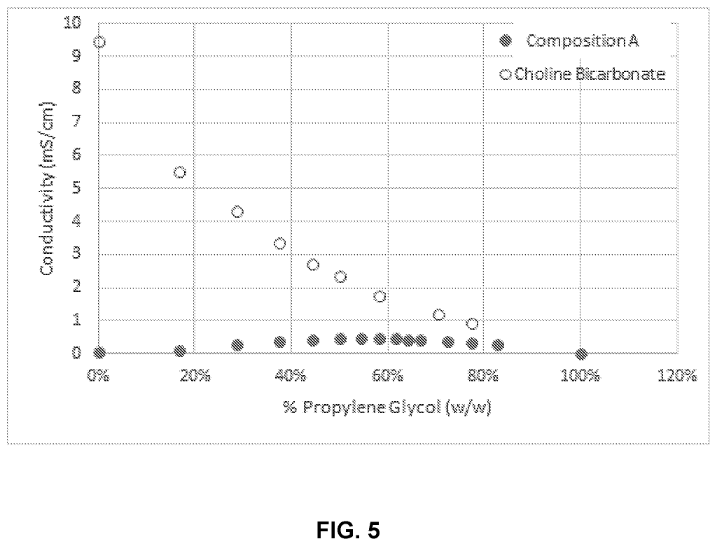

Results for geranic acid are shown in Table 1 and FIG. 3. Results for choline are shown in Table 2 and FIG. 4. Conductivity was also assessed between choline (bicarbonate) and Composition A (FIG. 5) in different amounts of % propylene glycol.

TABLE 1
Propylene glycol solubility, miscibility,
and conductivity with geranic acid

| Geranic | Propylene | Conductivity | |
|---|---|---|---|
| Acid % | Glycol % | (mS/cm) | Appearance |
| 100% | 0% | 0.00000 | Clear solution |
| 83% | 17% | 0.00000 | Clear solution |
| 71% | 29% | 0.00008 | Clear solution |
| 56% | 44% | 0.00008 | Clear solution |
| 36% | 64% | 0.00009 | Clear solution |

TABLE 2
Propylene glycol solubility, miscibility,
and conductivity with choline

| Choline | | | |
|---|---|---|---|
| Bicarbonate | Propylene | Conductivity | |
| % | Glycol % | (mS/cm) | Appearance |
| 100% | 0% | 9.470 | Clear solution |
| 83% | 17% | 5.540 | Clear solution |
| 71% | 29% | 4.320 | Clear solution |
| 63% | 38% | 3.360 | Clear solution |
| 56% | 44% | 2.710 | Clear solution |
| 50% | 50% | 2.350 | Clear solution |
| 42% | 58% | 1.738 | Clear solution |
| 29% | 71% | 1.186 | Clear solution |
| 23% | 77% | 0.916 | Clear solution |

Ethanol: Samples were made by first starting with 5 grams of each raw material; geranic acid first then repeated with choline (bicarbonate). In summary 1-3 grams of propylene glycol was added to the raw material being tested and mixed. A visual observation was made along with a conductivity measurement.

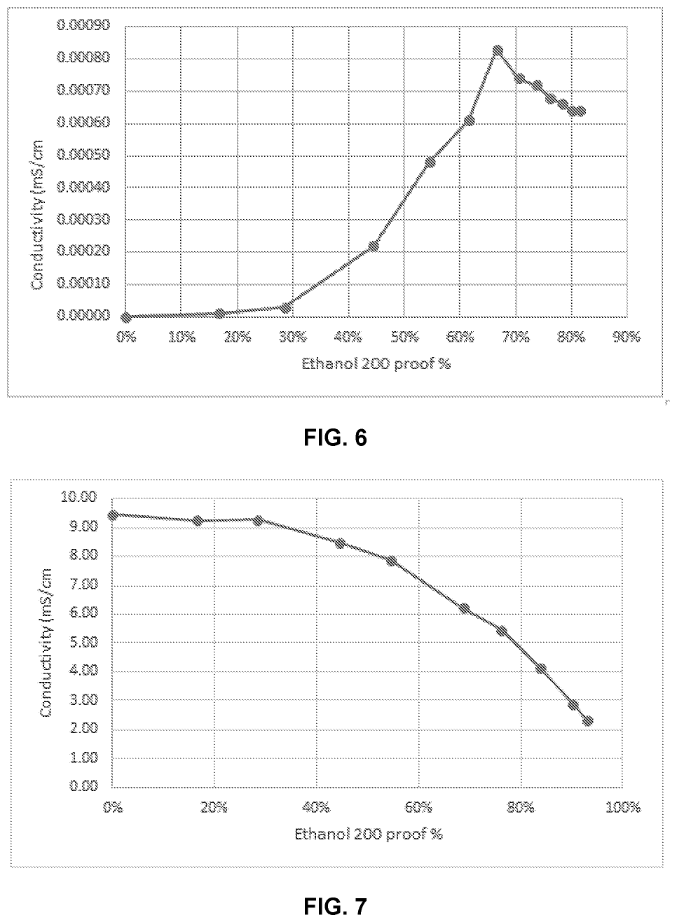

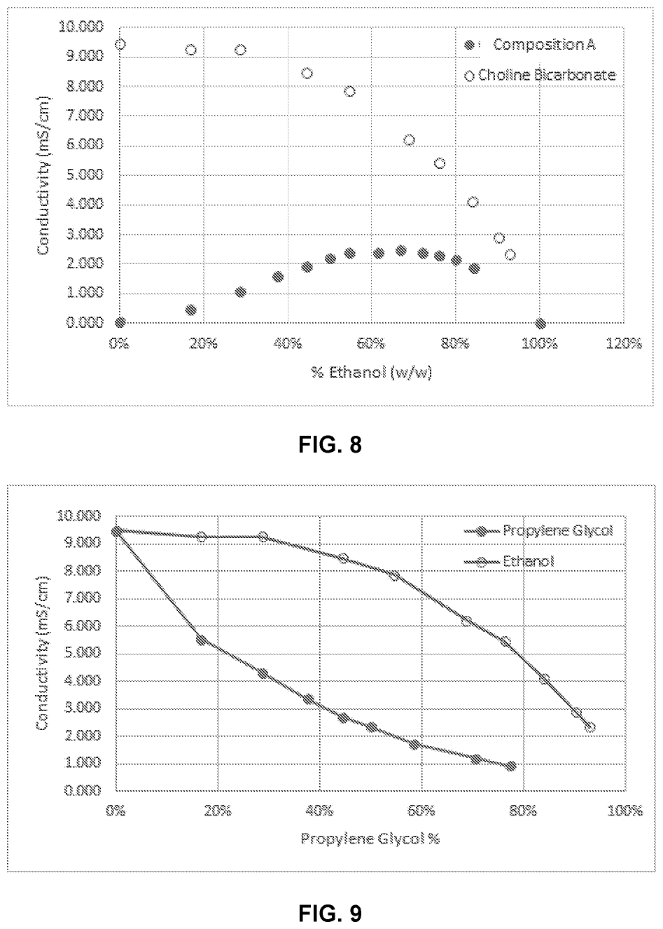

Results for geranic acid are shown in Table 3 and FIG. 6. Results for choline are shown in Table 4 and FIG. 7. Conductivity was also assessed between choline (bicarbonate) and Composition A (FIG. 8) in different amounts of % propylene glycol.

TABLE 3
Ethanol solubility, miscibility,
and conductivity with geranic acid

| Geranic | Ethanol 200 | Conductivity | |
|---|---|---|---|
| Acid % | proof % | (mS/cm) | Appearance |
| 100% | 0% | 0.00000 | Clear solution |
| 83% | 17% | 0.00001 | Clear solution |
| 71% | 29% | 0.00003 | Clear solution |
| 56% | 44% | 0.00022 | Clear solution |
| 45% | 55% | 0.00048 | Clear solution |
| 38% | 62% | 0.00061 | Clear solution |
| 33% | 67% | 0.00083 | Clear solution |
| 29% | 71% | 0.00074 | Clear solution |
| 26% | 74% | 0.00072 | Clear solution |
| 24% | 76% | 0.00068 | Clear solution |
| 22% | 78% | 0.00066 | Clear solution |
| 20% | 80% | 0.00064 | Clear solution |
| 19% | 81% | 0.00064 | Clear solution |

TABLE 4
Ethanol solubility, miscibility, and conductivity
with choline

| Choline | | | |
|---|---|---|---|
| Bicarbonate | Ethanol 200 | Conductivity | |
| % | proof % | (mS/cm) | Appearance |
| 100% | 0% | 9.47 | Clear solution |
| 83% | 17% | 9.26 | Clear solution |
| 71% | 29% | 9.27 | Clear solution |
| 56% | 44% | 8.49 | Clear solution |
| 45% | 55% | 7.88 | Clear solution |
| 31% | 69% | 6.22 | Clear solution |
| 24% | 76% | 5.45 | Clear solution |
| 16% | 84% | 4.12 | Clear solution |
| 10% | 90% | 2.89 | Clear solution |
| 7% | 93% | 2.33 | Clear solution |

Diisopropyl adipate: Samples were made by first starting with 5 grams of each raw material; geranic acid first then repeated with choline (bicarbonate). In summary, 1-3 grams of propylene glycol was added to the raw material being tested and mixed. A visual observation was made along with a conductivity measurement. Note: this study was abbreviated as the diisopropyl adipate was starting to degrade the plastic housing of the conductivity probe. When geranic acid continued to have zero conductivity the study was stopped, and when the choline experiment turned turbid the experiment was stopped.

TABLE 5
Diisopropyl adipate solubility, miscibility, and
conductivity with geranic acid

| Geranic | Diisopropyl | Conductivity | |
|---|---|---|---|
| Acid % | Adipate % | (mS/cm) | Appearance |
| 100.00% | 0.00% | 0 | Clear solution |
| 83.33% | 16.67% | 0 | Clear solution |
| 71.43% | 28.57% | 0 | Clear solution |

TABLE 6
Diisopropyl adipate solubility, miscibility, and
conductivity with choline

| Choline | Diisopropyl | Conductivity | |
|---|---|---|---|
| Bicarbonate% | Adipate% | (mS/cm) | Appearance |
| 100.00% | 0.00% | 9.47 | Clear solution |
| 83.33% | 16.67% | 6.29 | Turbid solution |

Additionally, the conductivity of choline in propylene glycol was compared to the conductivity of choline in ethanol (FIG. 9)

### Example 7: Solubility, Miscibility, and Conductivity of Composition a in Solvents and Solvent Blends

The solvents investigated included: water, propylene glycol, glycerin, PEG400, ethanol, diisopropyl adipate, mineral oil, propylene glycol/ethanol/water, and glycerin/PEGG400/water. Concentrations were titrated starting with 100% Composition A to about 80% of the solvent of interest (20% Composition A). Observations were taken at each concentration. Conductivity measurements were taken at each titrated point for information only at this stage. Scales on subsequent graphs are appropriate for each solvent's values.

Mineral oil: Mineral Oil was investigated by initially making a 50:50 (w/w) product with mineral oil and Composition A. The two products were not miscible. Two separate phases were produced. No further work was done with mineral oil at this time. No conductivity measurements were taken.

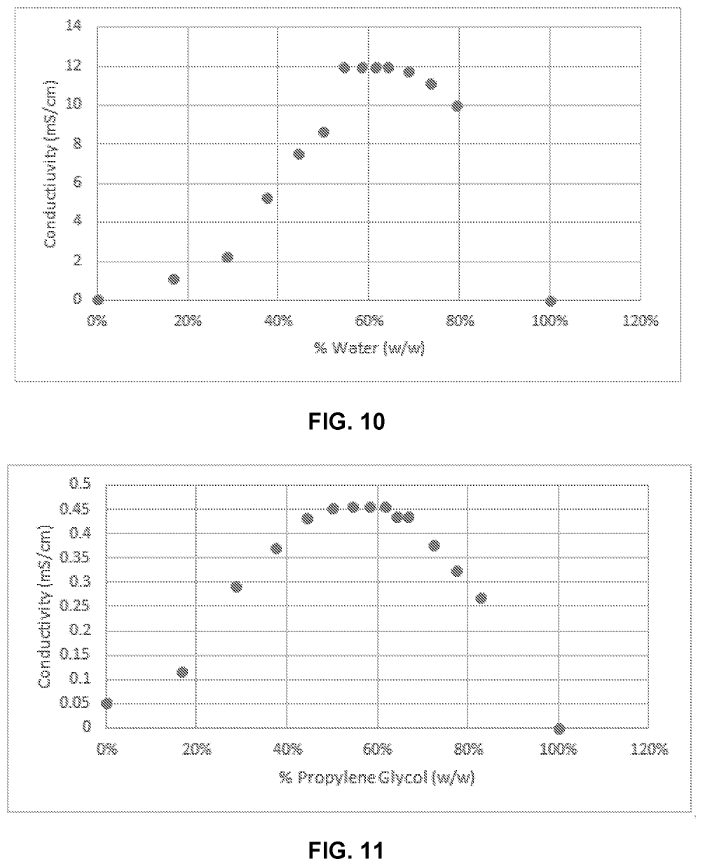

Water: Samples were made by first starting with 5 grams of Composition A. 1-3 grams of water was added and mixed. A visual observation was made along with a conductivity measurement. The process was continued until ˜25 grams of product was reached. The resulting product was stored for later evaluation. Results are shown in Table 7 and FIG. 10.

TABLE 7
Water solubility, miscibility, and conductivity

| Composition | Water | Conductivity | |
|---|---|---|---|
| A % | % | (mS/cm) | Appearance |
| 100% | 0% | 0.0519 | Clear-light yellow gel |
| 83% | 17% | 1.1156 | Turbid-Slightly |
| | | | Hazy Thick Gel |
| 71% | 29% | 2.2400 | Turbid-Thick Gel |
| 63% | 38% | 5.2700 | Turbid-Thick Hazy Gel |
| 56% | 44% | 7.5300 | Turbid-Thinned |
| 50% | 50% | 8.6800 | Turbid-Thinned |
| 45% | 55% | 11.9500 | Turbid-Slightly Hazy |
| | | | Thin |
| 42% | 58% | 11.9800 | Turbid-Hazy Thin |
| 38% | 62% | 11.9800 | Turbid-Slightly Hazy |
| 36% | 64% | 11.9900 | Clear |
| 31% | 69% | 11.7800 | Clear |
| 26% | 74% | 11.0900 | Clear |
| 21% | 79% | 9.9700 | Turbid-Hazy |
| | 100% | 0.0091 | Clear |

Propylene Glycol: Samples were made by first starting with 5 grams of Composition A. 1-3 grams of propylene glycol was added and mixed. A visual observation was made along with a conductivity measurement. The process was continued until ˜25 grams of product was reached. The resulting product was stored for later evaluation. Results are shown in Table 8 and FIG. 11.

TABLE 8
Propylene glycol solubility, miscibility,
and conductivity

| Composition | Propylene | Conductivity | |
|---|---|---|---|
| A % | Glycol % | (mS/cm) | Appearance |
| 100% | 0% | 0.0519 | Clear Solution |
| 83% | 17% | 0.1179 | Clear Solution |
| 71% | 29% | 0.291 | Clear Solution |
| 63% | 38% | 0.371 | Clear Solution |
| 56% | 44% | 0.432 | Clear Solution |
| 50% | 50% | 0.453 | Clear Solution |
| 45% | 55% | 0.456 | Clear Solution |
| 42% | 58% | 0.456 | Clear Solution |
| 38% | 62% | 0.457 | Clear Solution |
| 36% | 64% | 0.436 | Clear Solution |
| 33% | 67% | 0.436 | Clear Solution |
| 28% | 72% | 0.377 | Clear Solution |
| 23% | 77% | 0.324 | Clear Solution |
| 17% | 83% | 0.269 | Clear Solution |
| | 100% | 0.000 | Clear Solution |

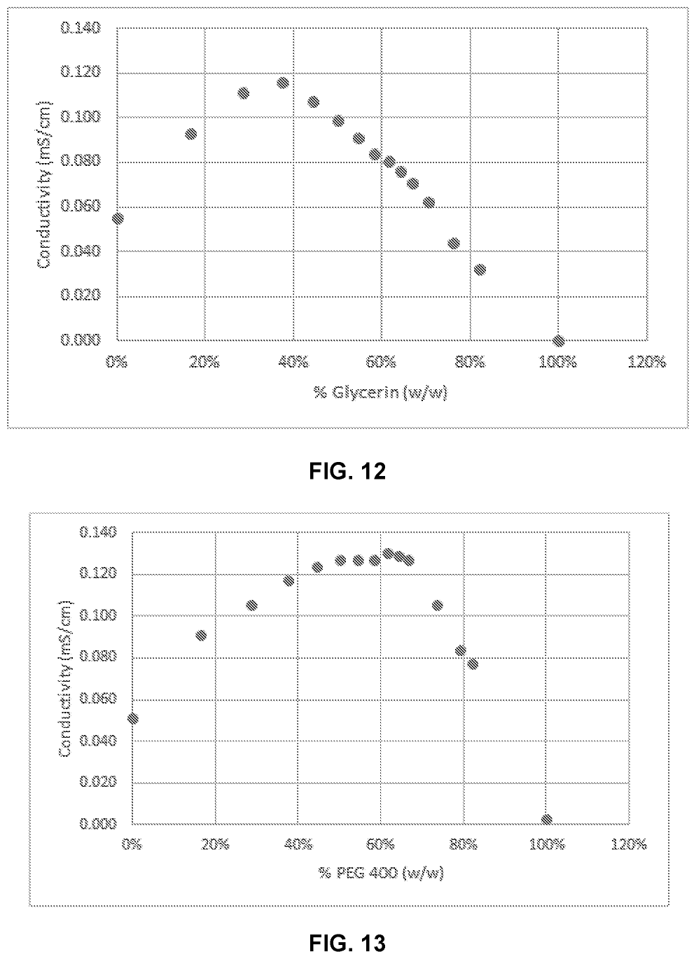

Glycerin: Samples were made by first starting with 5 grams of Composition A. 1-3 grams of glycerin was added and mixed. A visual observation was made along with a conductivity measurement. The process was continued until ˜25 grams of product was reached. The resulting product was stored for later evaluation. Results are shown in Table 9 and FIG. 12.

TABLE 9
Glycerin solubility, miscibility, and conductivity

| Composition | Glycerin | Conductivity | |
|---|---|---|---|
| A % | % | (mS/cm) | Appearance |
| 100% | 0% | 0.055 | Clear Solution |
| 83% | 17% | 0.093 | Clear Solution |
| 71% | 29% | 0.111 | Clear Solution |
| 63% | 38% | 0.116 | Clear Solution |
| 56% | 44% | 0.108 | Clear Solution |
| 50% | 50% | 0.099 | Clear Solution |
| 45% | 55% | 0.091 | Clear Solution |
| 42% | 58% | 0.084 | Clear Solution |
| 38% | 62% | 0.081 | Clear Solution |
| 36% | 64% | 0.076 | Clear Solution |
| 33% | 67% | 0.071 | Clear Solution |
| 29% | 71% | 0.062 | Clear Solution |
| 24% | 76% | 0.044 | Clear Solution |
| 18% | 82% | 0.032 | Clear to Turbid |
| | | | Solution |
| | 100% | 0.000 | Clear Solution |

PEG400: Samples were made by first starting with 5 grams of Composition A. 1-3 grams of PEG 400 was added and mixed. A visual observation was made along with a conductivity measurement. The process was continued until ˜25 grams of product was reached. The resulting product was stored for later evaluation. Results are shown in Table 10 and FIG. 13.

TABLE 10
PEG400 solubility, miscibility, and conductivity

| Composition | PEG400 | Conductivity | |
|---|---|---|---|
| A % | % | (mS/cm) | Appearance |
| 100% | 0% | 0.051 | Clear Solution |
| 83% | 17% | 0.091 | Clear Solution |
| 71% | 29% | 0.106 | Clear Solution |
| 63% | 38% | 0.117 | Clear Solution |
| 56% | 44% | 0.124 | Clear Solution |
| 50% | 50% | 0.127 | Clear Solution |
| 45% | 55% | 0.127 | Clear Solution |
| 42% | 58% | 0.127 | Clear Solution |
| 38% | 62% | 0.131 | Clear Solution |
| 36% | 64% | 0.129 | Clear Solution |
| 33% | 67% | 0.127 | Clear Solution |
| 26% | 74% | 0.105 | Clear Solution |
| 21% | 79% | 0.084 | Clear Solution |
| 18% | 82% | 0.077 | Clear Solution |
| 0% | 100% | 0.003 | Clear Solution |

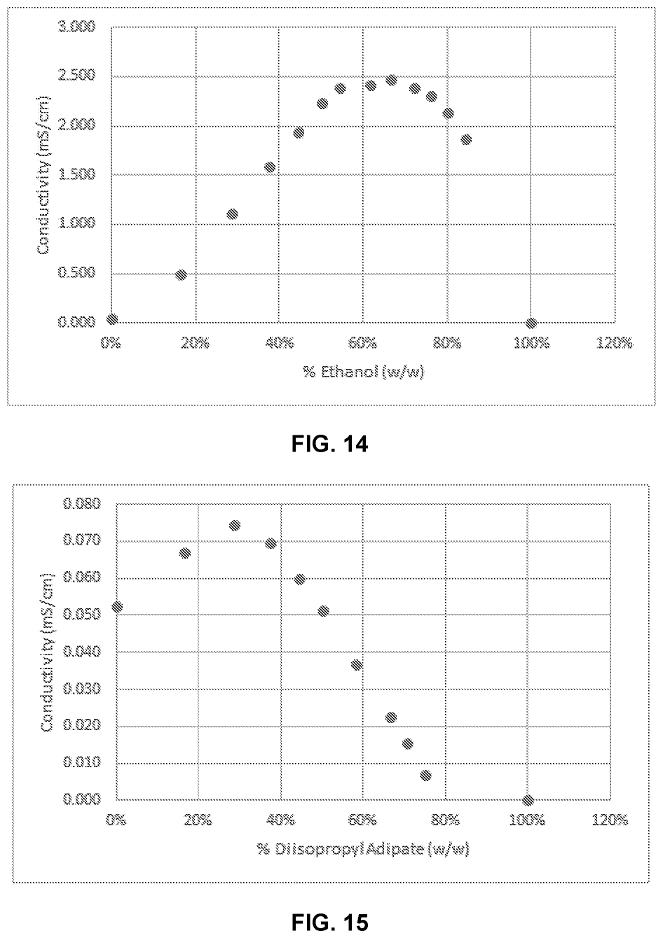

Ethanol: Samples were made by first starting with 5 grams of Composition A. 1-3 grams of ethanol was added and mixed. A visual observation was made along with a conductivity measurement. The process was continued until ˜25 grams of product was reached. The resulting product was stored for later evaluation. Results are shown in Table 11 and FIG. 14.

TABLE 11
Ethanol solubility, miscibility, and conductivity

| Composition | Ethanol | Conductivity | |
|---|---|---|---|
| A % | % | (mS/cm) | Appearance |
| 100% | 0% | 0.052 | Clear Solution |
| 83% | 17% | 0.499 | Clear Solution |
| 71% | 29% | 1.112 | Clear Solution |
| 63% | 38% | 1.581 | Clear Solution |
| 56% | 44% | 1.931 | Clear Solution |
| 50% | 50% | 2.230 | Clear Solution |
| 45% | 55% | 2.380 | Clear Solution |
| 38% | 62% | 2.410 | Clear Solution |
| 33% | 67% | 2.470 | Clear Solution |
| 28% | 72% | 2.390 | Clear Solution |
| 24% | 76% | 2.300 | Clear Solution |
| 20% | 80% | 2.140 | Clear Solution |
| 16% | 84% | 1.865 | Clear Solution |
| | 100% | 0.009 | Clear Solution |

Diisopropyl adipate: Samples were made by first starting with 5 grams of Composition A. 1-3 grams of diisopropyl adipate was added and mixed. A visual observation was made along with a conductivity measurement. The process was continued until ˜25 grams of product was reached. The resulting product was stored for later evaluation. Results are shown in Table 12 and FIG. 15.

TABLE 12
Diisopropyl adipate solubility, miscibility,
and conductivity

| Composition | DIA | Conductivity | |
|---|---|---|---|
| A % | % | (mS/cm) | Appearance |
| 100% | 0% | 0.052 | Clear Solution |
| 83% | 17% | 0.067 | Clear Solution |
| 71% | 29% | 0.075 | Clear Solution |
| 63% | 38% | 0.070 | Clear Solution |
| 56% | 44% | 0.060 | Clear Solution |
| 50% | 50% | 0.051 | Clear Solution |
| 42% | 58% | 0.037 | Clear Solution |
| 33% | 67% | 0.022 | Clear Solution |
| 29% | 71% | 0.015 | Clear Solution |
| 25% | 75% | 0.007 | Clear Solution |
| | 100% | 0.000 | Clear Solution |

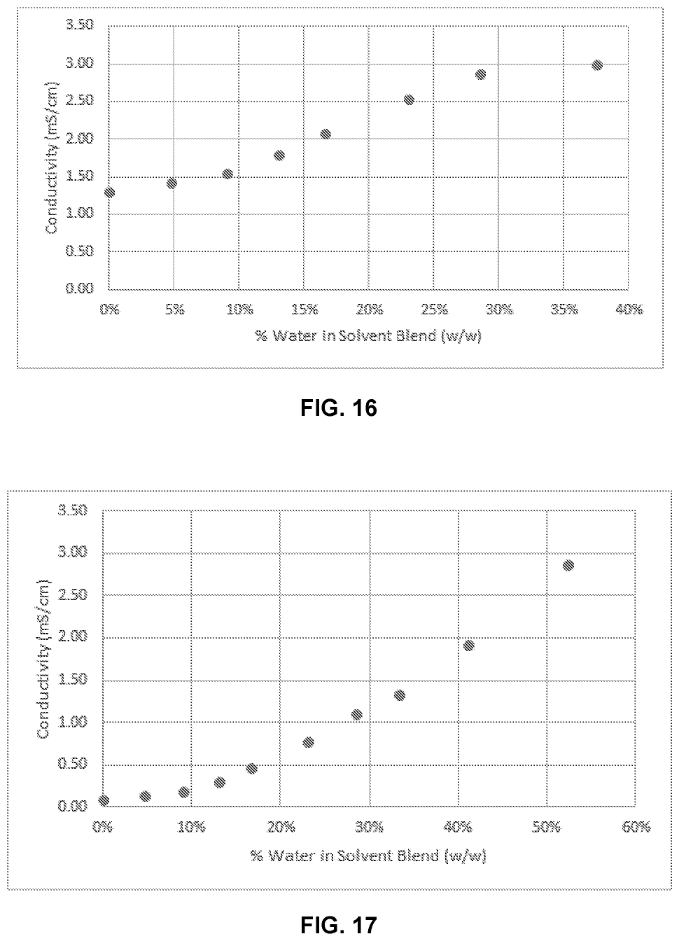

Propylene glycol, ethanol, Composition A, and water blend: Samples were made by first starting with 3 grams of Composition A, 3.5 grams of propylene glycol, and 3.5 grams of ethanol, mixed until clear. 1-3 grams of water was added and mixed. A visual observation was made along with a conductivity measurement. The process was continued until ˜16 grams of product was reached. The resulting product was stored for later evaluation. Results are shown in Table 13 and FIG. 16.

TABLE 13
Solubility, miscibility, and conductivity
of a blend of propylene glycol,
ethanol, Composition A, and water

| Com- | | Et- | | Con- | |
|---|---|---|---|---|---|
| position | Propylene | hanol | Water | ductivity | |
| A % | Glycol % | % | % | (mS/cm) | Appearance |
| 30% | 35% | 35% | 0% | 1.30 | Clear Solution |
| 29% | 33% | 33% | 5% | 1.42 | Clear Solution |
| 27% | 32% | 32% | 9% | 1.56 | Clear Solution |
| 26% | 30% | 30% | 13% | 1.80 | Clear Solution |
| 25% | 29% | 29% | 17% | 2.08 | Clear Solution |
| 23% | 27% | 27% | 23% | 2.54 | Clear Solution |
| 21% | 25% | 25% | 29% | 2.86 | Clear Solution |
| 19% | 22% | 22% | 38% | 3.00 | Clear Solution |

Glycerin, PEG400, Composition A, and water blend: Samples were made by first starting with 3 grams of Composition A, 3.5 grams of glycerin, and 3.5 grams of PEG400, mixed until clear. 1-3 grams of water was added and mixed. A visual observation was made along with a conductivity measurement. The process was continued until ˜20 grams of product was reached. The resulting product was stored for later evaluation. Results are shown in Table 14 and FIG. 17.

TABLE 14
Solubility, miscibility, and conductivity of a blend of glycerin,
PEG400, Composition A, and water

| Composition | Gly- | PEG400 | Water | Conductivity | |
|---|---|---|---|---|---|
| A % | cerin % | % | % | (mS/cm) | Appearance |
| 30% | 35% | 35% | 0% | 0.09 | Clear Solution |
| 29% | 33% | 33% | 5% | 0.14 | Clear Solution |
| 27% | 32% | 32% | 9% | 0.19 | Clear Solution |
| 26% | 30% | 30% | 13% | 0.30 | Clear Solution |
| 25% | 29% | 29% | 17% | 0.46 | Clear Solution |
| 23% | 27% | 27% | 23% | 0.78 | Clear Solution |
| 21% | 25% | 25% | 29% | 1.09 | Clear Solution |
| 20% | 23% | 23% | 33% | 1.33 | Clear Solution |
| 18% | 21% | 21% | 41% | 1.92 | Clear Solution |
| 14% | 17% | 17% | 52% | 2.86 | Turbid |

### Example 8: Skin Flux Assay of a Choline:Geranic Acid Composition in a 1:2 Molar Ratio Compared to Choline and Geranic Acid Individually

Skin permeation was measured using a static Franz cell setup. Dermatomed human cadaver skin samples procured from NY fire fighters skin bank were used. The skin sample thickness was between 0.29-0.59 mm. The 9-mm Franz cell cells had a receptor chamber volume of 4 ml and a donor chamber volume of 2 ml. The nominal skin contact area for the formulations was 0.64 cm2.

Approximately 100 μl from the following 5 different samples were added to the donor compartment of 15 cells respectively:
1. 20% Composition A (Choline:Geranic Acid in a molar ratio 1:2) in 80% diisopropyl adipate (N=3, Donor 1)
2. Composition A, Choline:Geranic Acid in a molar ratio 1:2 (N=3, Donor 1)
3. 80% Choline bicarbonate in water (N=3, Donor 1)
4. Geranic Acid (N=3, Donor 1)
5. 20% Composition A (Choline:Geranic Acid in a molar ratio 1:2) in 80% diisopropyl adipate (N=3, Donor 2)

The receptor chamber contained 4 ml of phosphate buffered saline at a pH 7.4. A magnetic stirrer was added to the receptor chamber of each cell. All 15 cells were placed in an oven held at 32° C. Samples were withdrawn from the receptor chamber at 0 h, 1 h, 4 h, 7 h, and 24 h. The concentration of choline and geranic acid in these samples was quantified by HPLC.

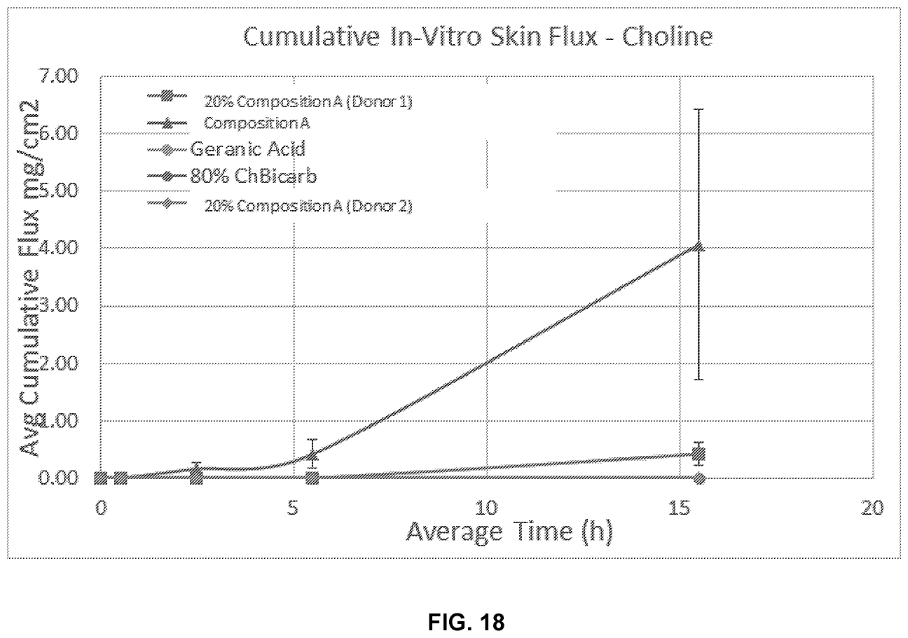

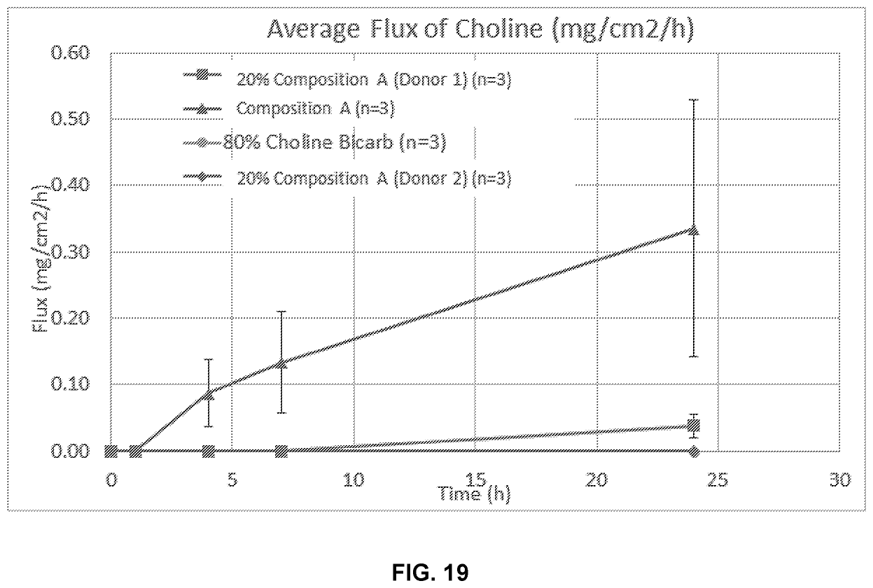

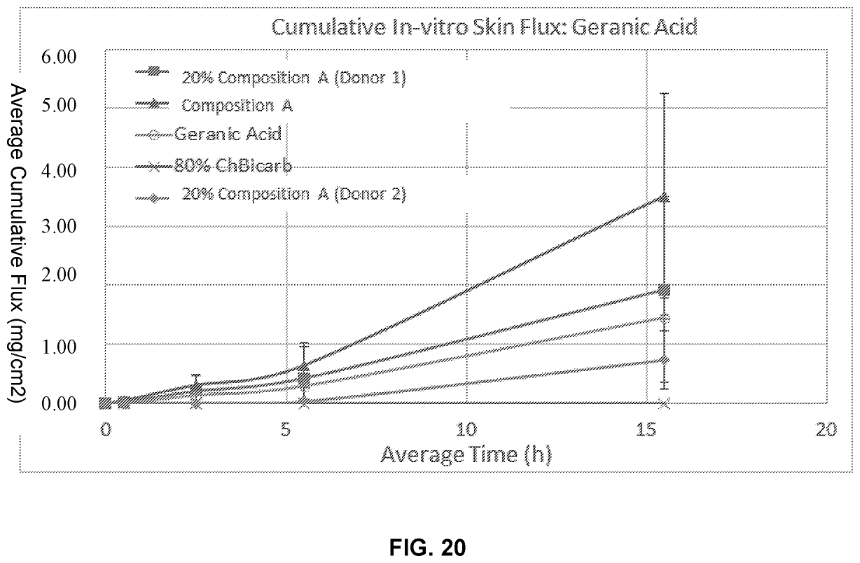

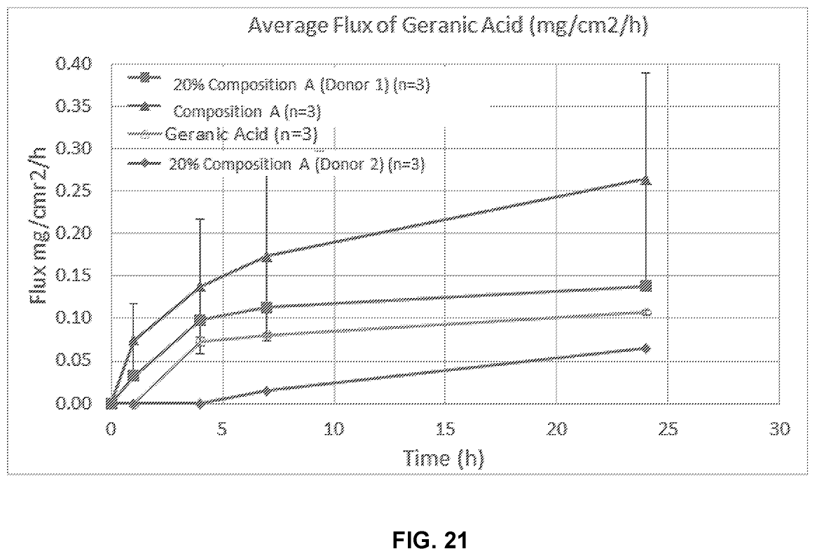

Cumulative and average skin flux of choline are shown in FIG. 18 and FIG. 19, while cumulative and average skin flux for geranic acid are shown in FIG. 20 and FIG. 21, respectively. The data demonstrate that the individual components had little to no permeability through the skin. However, Composition A showed a dramatic increase in the permeation of both components of Composition A. The average flux of choline and geranic acid from Composition A at 24 h was 0.34 and 0.26 mg/cm²/h which was significantly higher than the flux from the individual components. The flux of Composition A decreased proportionally when diluted to 20% using diisopropyl adipate as a solvent.

### Example 9: Skin Flux Assay of a Choline:Geranic Acid Composition in a 1:2 Molar Ratio Compared to a Choline:Geranic Acid Composition in a 1:1 Molar Ratio

Skin permeation was measured using a static Franz cell setup. Dermatomed human cadaver skin samples procured from NY fire fighters skin bank were used. The skin sample thickness was between 0.29-0.59 mm. The 9-mm Franz cell cells had a receptor chamber volume of 4 ml and a donor chamber volume of 2 ml. The nominal skin contact area for the formulations was 0.64 cm².

Approximately 100 μl from the following 5 different samples were added to the donor compartment of 15 cells respectively:
1. Composition B (Choline:Geranic Acid in a molar ratio 1:1) (N=3, Donor 1)
2. 40% Composition B, (Choline:Geranic Acid in a molar ratio 1:1) in Milli-Q® water (N=3, Donor 1)
3. 40% Composition A, (Choline:Geranic Acid in a molar ratio 1:2) in Milli-Q® water (N=3, Donor 1)
4. 40% Composition A, (Choline:Geranic Acid in a molar ratio 1:1) in diisopropyl adipate (N=3, Donor 1)
5. 40% Composition B, (Choline:Geranic Acid in a molar ratio 1:2) in diisopropyl adipate (N=3, Donor 1)

The receptor chamber contained 4 ml of phosphate buffered saline at a pH 7.4. A magnetic stirrer was added to the receptor chamber of each cell. All 15 cells were placed in an oven held at 32° C. Samples were withdrawn from the receptor chamber at 0 h, 1 h, 4 h, 7 h, and 24 h. The concentration of choline and geranic acid in these samples was quantified by HPLC.

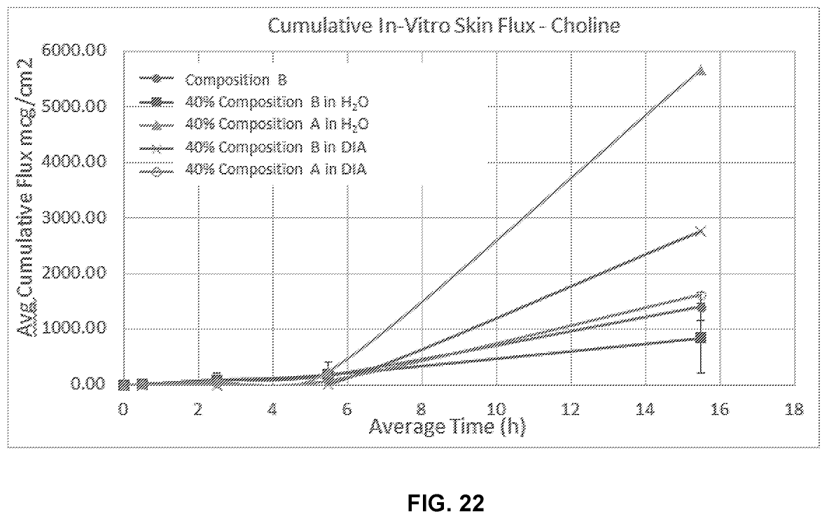

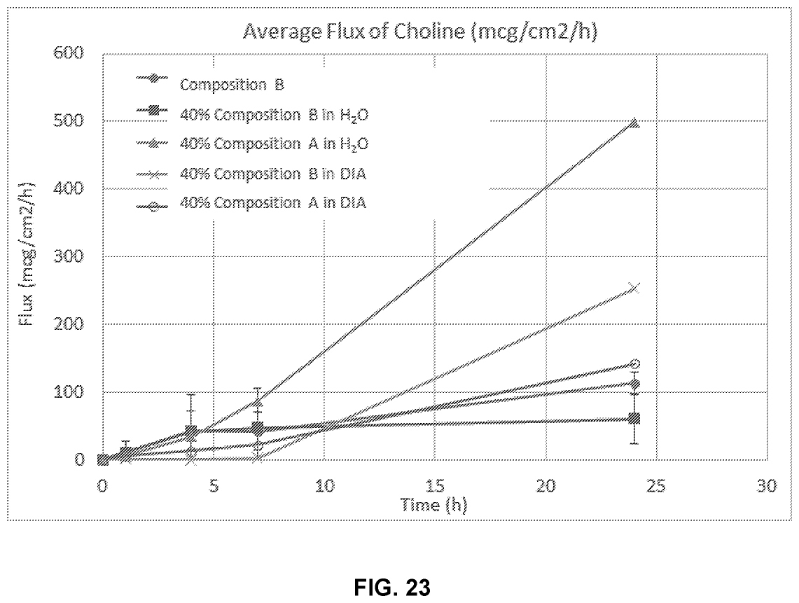

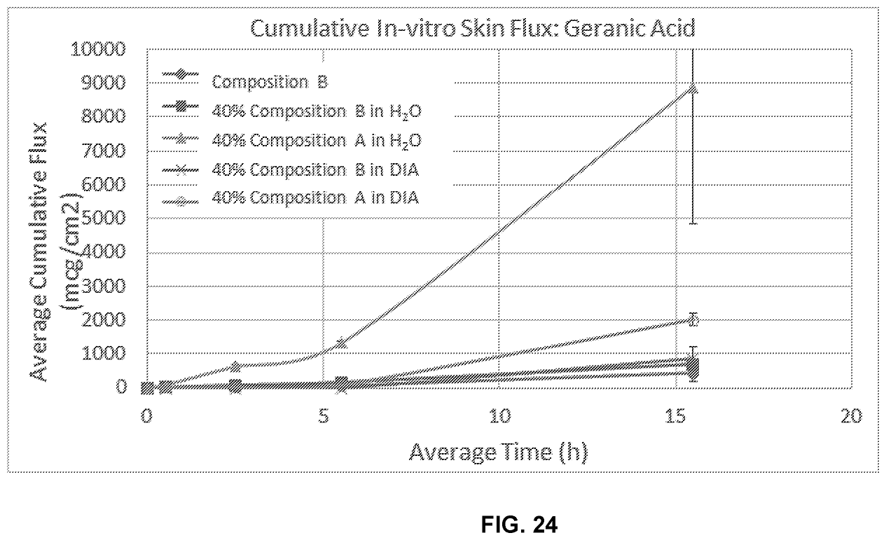

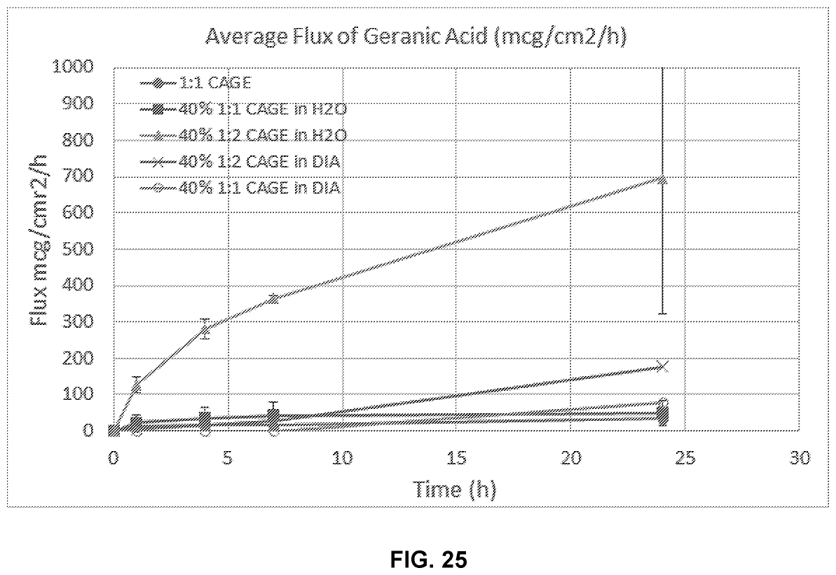

Cumulative and average skin flux of choline are shown in FIG. 22 and FIG. 23, while cumulative and average skin flux for geranic acid are shown in FIG. 24 and FIG. 25, respectively. The data demonstrated that both compositions resulted in an increase in permeation of both compounds compared to the individual components. The average flux of choline and geranic acid from Composition B at 24 h was 0.11 and 0.03 mg/cm²/h. The flux of choline and geranic acid from both compositions was higher when diluted with water compared to dilution with diisopropyl adipate. These results demonstrated that it was feasible to get meaningful concentrations of both compounds in the skin when formulated as Composition A or 2 diluted in common solvents such as water.

### Example 10: Formulation Study

In order to leverage the previous data generated with solvent systems and the relative conductivity of each with Composition A, a gel system was chosen to move forward with. The first gelling agents investigated were cellulose gels since they are non-ionic. Three gelling agents were looked at: Natrosol 250 HEX Pharma, Hydroxyethyl cellulose (HEC); Klucel MF Pharm, Hydroxypropyl cellulose (HPC); and Benecel E5 Pharm, Hydroxypropylmethyl cellulose (HPMC).

Single solvents were looked at for gelling potential and resulting conductivity. The conductivity and gelling ability of a single solvent and a gelling agent are shown in Table 15. Hydroxypropyl cellulose was the most consistent gelling agent for some of the solvents which were previously worked with.

TABLE 15
Conductivity and gelling ability results

| Ingredient + Gelling Agent | | |
|---|---|---|
| Gelled (y/n) | Conductivity | |
| Water + HEC | y | 0.1515 |
| Water + HPC | y | 0.01698 |
| Water + HPMC | n | na |
| Diisopropyl adipate + HEC | n | na |
| Diisopropyl adipate + HPC | n | na |
| Diisopropyl adipate + HPMC | n | na |
| PEG 400 + HEC | n | na |
| PEG 400 + HPC | n | na |
| PEG 400 + HPMC | n | na |
| Propylene Glycol + HEC | n | na |
| Propylene Glycol + HPC | y | 0.00024 |
| Propylene Glycol + HPMC | n | na |
| Ethanol + HEC | n | na |
| Ethanol + HPC | y | 0.00191 |
| Ethanol + HPMC | n | na |

A brief miscibility study was performed using 50/50 mixtures and diisopropyl adipate and various solvents, and the results are shown in Table 16.

TABLE 16
Diisopropyl adipate miscibility

| | Miscible |
|---|---|
| Ingredient Combination | (y/n) |
| Diisoproply Adipate + Water | n |
| Diisoproply Adipate + Propylene Glycol | n |
| Diisoproply Adipate + Ethanol | y |
| Diisoproply Adipate + Glycerin | n |

Since ethanol was the only solvent studied which was miscible with the diisopropyl adipate, some concentration of ethanol was blended into most formulations in order to generate a true solution. A concentration of 20% diisopropyl adipate was used as this was the maximum allowable level according to the FDA inactive ingredient database (HD). A study with propylene glycol and glycerin were performed to see how little ethanol was needed to still result in a clear solution, and the results are shown in Table 17. A similar study was repeated with glycerin, and the results are shown in Table 18.

TABLE 17
Propylene glycol solubility study

| Diisopropyl | Ethanol | Propylene | Miscible | |
|---|---|---|---|---|
| Adipate (g) | (g) | Glycol (g) | (y/n) | Conductivity |
| 20 | 10 | 50 | y | 0.00013 |
| 20 | 20 | 40 | y | 0.00024 |
| 20 | 30 | 20 | y | 0.00047 |
| 20 | 40 | 20 | y | 0.00081 |
| 20 | 50 | 10 | y | 0.00068 |

TABLE 18
Glycerin solubility study

| Diisopropyl | Ethanol | Glycerin | Miscible | |
|---|---|---|---|---|
| Adipate (g) | (g) | (g) | (y/n) | Conductivity |
| 20 | 10 | 50 | n | na |
| 20 | 20 | 40 | n | na |
| 20 | 30 | 30 | y | 0.0001 |
| 20 | 40 | 20 | y | 0.00024 |
| 20 | 35 | 25 | y | 0.00017 |

The glycerin formulation was further evaluated for an appropriate gelling agent which would gel the diisopropyl adipate/ethanol/glycerin blend, and the results are shown in Table 19. HPC was the gelling agent for the diisopropyl adipate/ethanol/glycerin blend.

TABLE 19
Glycerin base gelling agent study

| | Gelled | Conduc- |
|---|---|---|
| Ingredient + Gelling Agent | (y/n) | tivity |
| Diisopropyl Adipate/Ethanol/Glycerin Blend + HEC | n | na |
| Diisopropyl Adipate/Ethanol/Glycerin Blend + HPC | y | 0.0022 |
| Diisopropyl Adipate/Ethanol/Glycerin Blend + HPMC | n | na |

A base formulation was chosen based on the assumption that 20% of Composition A would be used to dilute the final base formula. The base formula used is illustrated in Table 20.

TABLE 20
Glycerin base formulation

| Ingredient | % w/w |
|---|---|
| Diisopropyl Adipate | 25% |
| Ethanol (200 proof) | 43% |
| Glycerin | 30% |
| Klucel (HPC) | 3% |

Initially a sample of choline/geranic acid (1:2) was used to verify compatibility of all the ingredients. This sample of choline/geranic acid (1:2) still had small amounts of water present as it was not evaporated off as it was with Composition A. The gel base was added stepwise to the choline/geranic acid and the conductivity was measured, and the results are shown in Table 21.

TABLE 21
Conductivity results of titrated glycerin formulation

| Choline/ | HPC | | |
|---|---|---|---|
| Geranic | Gel | | |
| Acid (1:2) | Base | Conductivity | |
| % | % | (mS/cm) | Appearance |
| 100% | 0% | 0.864 | Yellow clear solution |
| 50% | 50% | 1.099 | Slight hazy, light yellow gel |
| 33% | 67% | 0.950 | Slight hazy, slight yellow gel |
| 25% | 75% | 0.768 | Slight hazy, slight yellow gel |
| 20% | 80% | 0.599 | Slight hazy, slight yellow gel |
| 17% | 83% | 0.528 | Slight hazy, slight yellow gel |

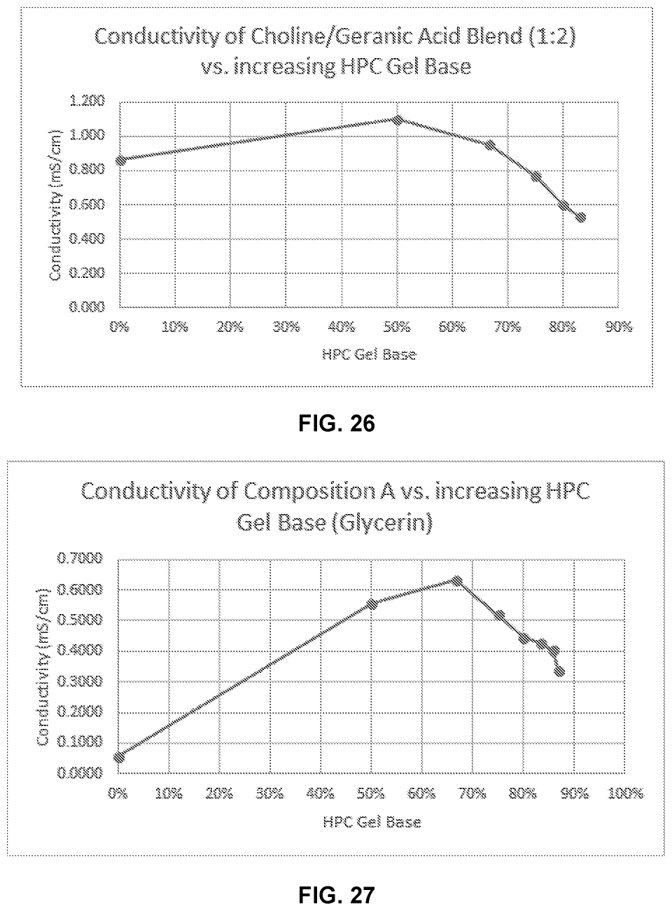

FIG. 26 and FIG. 27 show conductivity of titrated glycerin formulations. No adverse effects seemed apparent and the final product appeared to be a viscous gel. The experiment was repeated with Composition A, and the results are shown in Table 22.

TABLE 22
Conductivity results of titrated glycerin formulation
(Composition A)

| | HPC | | |
|---|---|---|---|
| | Gel | | |
| Composition A | Base | Conductivity | |
| % | % | (mS/cm) | Appearance |
| 100% | 0% | 0.0552 | Yellow clear viscous liquid |
| 50% | 50% | 0.557 | Slight hazy, yellow gel |
| 33% | 67% | 0.634 | Slight hazy, light yellow gel |
| 25% | 75% | 0.518 | Slight hazy, light yellow gel |
| 20% | 80% | 0.446 | Slight hazy, light yellow gel |
| 17% | 83% | 0.427 | Slight hazy, light yellow gel |
| 14% | 86% | 0.402 | Slight hazy, light yellow gel |
| 13% | 87% | 0.337 | Slight hazy, light yellow gel |

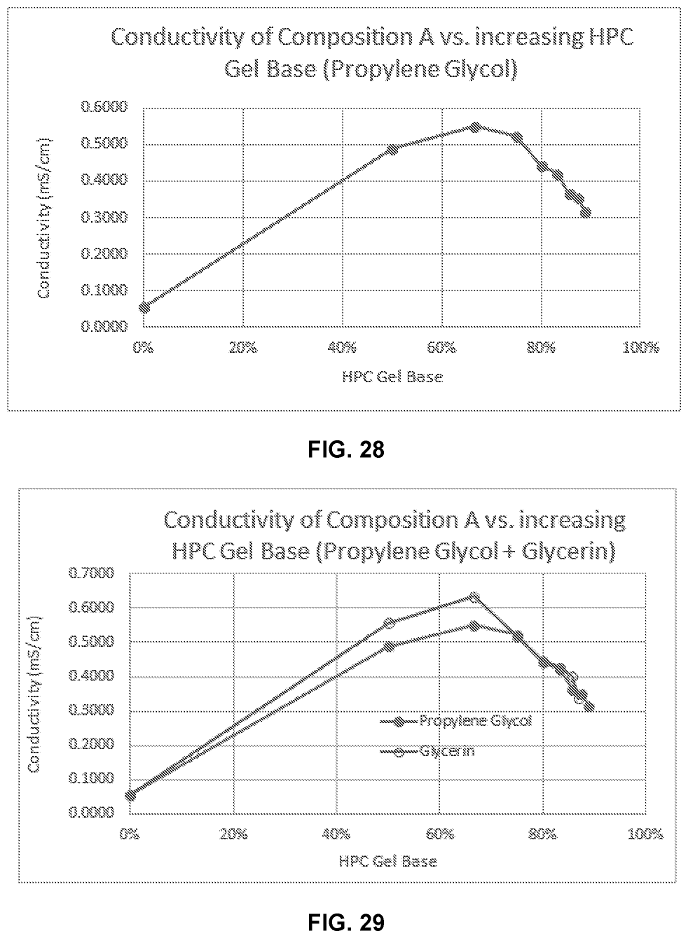

A similar experiment with the propylene glycol formulation was performed. The results are shown in Table 23, Table 24, and FIG. 28.

TABLE 23
Propylene glycol base formulation.

| Ingredient | % w/w |
|---|---|
| Diisopropyl Adipate | 25% |
| Ethanol (200 proof) | 13% |
| Propylene Glycol | 60% |
| Klucel (HPC) | 3% |

TABLE 24
Conductivity results of titrated propylene glycol formulation
(Composition A)

| | HPC | | |
|---|---|---|---|
| | Gel | | |
| Composition A | Base | Conductivity | |
| % | % | (mS/cm) | Appearance |
| 100% | 0% | 0.0552 | Yellow clear viscous liquid |
| 50% | 50% | 0.488 | Yellow clear gel |
| 33% | 67% | 0.551 | Light, yellow clear gel |
| 25% | 75% | 0.523 | Light, yellow clear gel |
| 20% | 80% | 0.443 | Slight yellow, clear gel |
| 17% | 83% | 0.420 | Slight yellow, clear gel |
| 14% | 86% | 0.364 | Slight yellow, clear gel |
| 13% | 88% | 0.352 | Slight yellow, clear gel |
| 11% | 89% | 0.315 | Slight yellow, clear gel |

The conductivity of the two formulations was compared (FIG. 29). The two formulations performed similarly, with both products having a similar max conductivity, with the overall conductivity of the propylene glycol product being lower.

### Example 11: Repeat Insult Patch Test for Skin Irritation and Sensitization Evaluation

A study was performed to assess the amount of skin irritation caused by Composition A when applied to the skin. 52 subjects were enrolled in this study ranging in age from 21 to 67.8 of the subjects were male and 44 were female. 44 of the subjects were Caucasian, 12 were Hispanic, and 2 were Asian. Of these 52 subjects, 50 completed the study.

Standards for inclusion in the study included:
Individuals who are not currently under a doctor's care.
Individuals free of any dermatological or systemic disorder which would interfere with the results.
Individuals free of any acute or chronic disease that might interfere with or increase the risk of study participation.

Standards for exclusion from the study included:
Individuals under 18 years of age.
Individuals who are currently under a doctor's care.
Individuals who are currently taking any medication (topical or systemic) that may mask or interfere with the test results.
Individuals with a history of any acute or chronic disease that might interfere with or increase the risk associated with study participation.
Individuals diagnosed with chronic skin allergies.
Female volunteers who indicate that they are pregnant or lactating.

Distilled water was used as a negative control. 0.2 mL of distilled water was dispensed onto the occlusive, hypoallergenic patch.

Composition A was applied as a thin layer directly onto a 2×2 cm designated area of skin on the subject's back and the covered with the occlusive, hypoallergenic patch. The subject was then dismissed with instructions not to wet or expose the test area to direct sunlight. After 24 hours the patches were removed by the subject at home.

This procedure was repeated until a series of nine consecutive 24 hour exposures had been made for every Monday, Wednesday, and Friday for three consecutive weeks.

If an adverse reaction occurred, the area of erythema and edema was measured. The edema was estimated by the evaluation of the skin with respect to the contour of the unaffected normal skin. Reactions were scored just before applications two through nine and the next test date following application nine. In most instances, this was approximately 24 hours after patch removal.

Subjects were then given a 10-14 day rest period after which a challenge or retest dose was applied once to a previously unexposed test site. The retest dose was equivalent to any one of the original nine exposures. Reactions were scored 24 and 48 hours after application (Table 25). Eight panelists displayed grade 1-3 irritation on the fourth through ninth evaluation days. No other adverse reactions of any kind were noted.

TABLE 25
Skin reactions to Composition A

| | | | | | | | | | | | | | Chall. | | |
|---|---|---|---|---|---|---|---|---|---|---|---|---|---|---|---|
| | Subject | | | Response | | | | | | | | | 24 | 48 | |
| No. | ID | RACE | SEX | 1 | 2 | 3 | 4 | 5 | 6 | 7 | 8 | 9 | HR | HR | Score |
| 1 | 35 8496 | H | M | 0 | 0 | 0 | 0 | 0 | 0 | 0 | 0 | 0 | 0 | 0 | 0.0 |
| 2 | 44 7255 | C | F | 0 | 0 | 0 | 0 | 0 | 0 | 0 | 0 | 0 | 0 | 0 | 0.0 |
| 3 | 44 9509 | C | F | 0 | 0 | 0 | 1 | 2 | 3 | D | D | D | 0 | 0 | 6.0 |
| 4 | 48 2063 | C | F | 0 | 0 | 0 | 1 | 3 | D | D | D | D | 0 | 0 | 4.0 |
| 5 | 48 6033 | H | M | 0 | 0 | 0 | 0 | 0 | 0 | 0 | 0 | 0 | 0 | 0 | 0.0 |
| 6 | 50 1289 | C | F | 0 | 0 | 0 | 0 | 1 | 3 | D | D | D | 0 | 0 | 4.0 |
| 7 | 52 0525 | C | F | 0 | 0 | 0 | 0 | 0 | 0 | 0 | 0 | 0 | 0 | 0 | 0.0 |
| 8 | 52 2026 | H | M | 0 | 0 | 0 | 1 | 1 | 3 | D | D | D | 0 | 0 | 5.0 |
| 9 | 54 0346 | C | F | 0 | 0 | 0 | 0 | 0 | 0 | 1 | 2 | 0 | 0 | 0 | 4.0 |
| 10 | 54 1758 | C | F | 0 | 0 | 0 | 0 | 0 | 0 | 0 | 0 | 0 | 0 | 0 | 0.0 |
| 11 | 54 4281 | C | M | 0 | 0 | 0 | 0 | 0 | 0 | 0 | 0 | 0 | 0 | 0 | 0.0 |
| 12 | 54 4323 | C | F | 0 | 0 | 0 | 0 | 0 | 0 | 0 | 0 | 0 | 0 | 0 | 0.0 |
| 13 | 54 7647 | C | F | 0 | 0 | 0 | 0 | 0 | 0 | 0 | 0 | 0 | 0 | 0 | 0.0 |
| 14 | 56 1236 | C | F | 0 | 0 | 0 | 0 | 0 | 0 | 0 | 0 | 0 | 0 | 0 | 0.0 |
| 15 | 56 1830 | C | F | 0 | 0 | 0 | 0 | 0 | 0 | 0 | 0 | 0 | 0 | 0 | 0.0 |
| 16 | 56 3141 | H | F | 0 | 0 | 0 | 0 | 0 | 0 | 0 | 0 | 0 | 0 | 0 | 0.0 |
| 17 | 56 3379 | C | F | 0 | 0 | 0 | 0 | 0 | 0 | 0 | 0 | 0 | 0 | 0 | 0.0 |
| 18 | 56 3465 | H | F | 0 | 0 | 0 | 0 | 0 | 0 | 0 | 0 | 0 | 0 | 0 | 0.0 |
| 19 | 56 4283 | H | F | 0 | 0 | 0 | 0 | 0 | 0 | 0 | 0 | 0 | 0 | 0 | 0.0 |
| 20 | 56 5529 | C | F | 0 | 0 | 0 | 0 | 0 | 0 | 0 | 0 | 0 | 0 | 0 | 0.0 |
| 21 | 56 6301 | C | F | 0 | 0 | 0 | 0 | 0 | 0 | 0 | 0 | 0 | 0 | 0 | 0.0 |
| 22 | 56 6648 | C | F | 0 | 0 | 0 | 0 | 0 | 0 | 0 | 0 | 0 | 0 | 0 | 0.0 |
| 23 | 56 8344 | H | F | 0 | 0 | 0 | 0 | 3 | D | D | D | D | 0 | 0 | 3.0 |
| 24 | 58 8233 | H | F | 0 | 0 | 0 | 1 | 2 | 3 | D | D | D | 0 | 0 | 6.0 |
| 25 | 60 8701 | C | F | 0 | 0 | 0 | 0 | 0 | 0 | 0 | 0 | 0 | 0 | 0 | 0.0 |
| 26 | 60 9466 | C | F | 0 | 0 | 0 | 0 | 0 | 0 | 0 | 0 | 0 | 0 | 0 | 0.0 |
| 27 | 62 1419 | C | F | 0 | 0 | 0 | 0 | 0 | 0 | 0 | 0 | 0 | 0 | 0 | 0.0 |
| 28 | 62 2435 | C | F | 0 | 0 | 0 | 0 | 0 | 0 | 0 | 0 | 0 | 0 | 0 | 0.0 |
| 29 | 62 8070 | H | F | 0 | 0 | 0 | 0 | 0 | 0 | 0 | 0 | 0 | 0 | 0 | 0.0 |
| 30 | 62 9431 | C | F | 0 | 0 | 0 | 0 | 0 | 0 | 0 | 0 | 0 | 0 | 0 | 0.0 |
| 31 | 64 0667 | C | F | 0 | 0 | 0 | 0 | 0 | 0 | 0 | 0 | 0 | 0 | 0 | 0.0 |
| 32 | 64 4506 | C | F | 0 | 0 | 0 | 0 | 0 | 0 | 0 | 0 | 0 | 0 | 0 | 0.0 |
| 33 | 64 4610 | C | F | 0 | 0 | 0 | 0 | 0 | 0 | 0 | 0 | 0 | 0 | 0 | 0.0 |
| 34 | 66 0508 | C | M | 0 | 0 | 0 | 0 | 0 | 0 | 0 | 0 | 0 | 0 | 0 | 0.0 |
| 35 | 66 8456 | C | F | 0 | 0 | 0 | 0 | 0 | 0 | 0 | 0 | 0 | 0 | 0 | 0.0 |
| 36 | 68 6060 | C | F | 0 | 0 | 0 | 0 | 0 | 0 | 0 | 0 | 0 | 0 | 0 | 0.0 |
| 37 | 70 6695 | C | F | 0 | 0 | Dc | Dc | Dc | Dc | Dc | Dc | Dc | Dc | Dc | N/A |
| 38 | 70 9766 | C | F | 0 | 0 | 0 | 0 | 0 | 0 | 0 | 0 | 0 | 0 | 0 | 0.0 |
| 39 | 72 3555 | C | F | 0 | 0 | 0 | 0 | 0 | 0 | 0 | 0 | 0 | 0 | 0 | 0.0 |
| 40 | 72 3637 | H | F | 0 | 0 | 0 | 0 | 0 | 0 | 0 | 0 | 0 | 0 | 0 | 0.0 |
| 41 | 72 4644 | C | F | 0 | 0 | 0 | 0 | 0 | 0 | 0 | 0 | 0 | 0 | 0 | 0.0 |
| 42 | 72 9050 | C | M | 0 | 0 | 0 | 0 | 0 | 0 | 0 | 0 | 0 | 0 | 0 | 0.0 |
| 43 | 73 6193 | H | F | 0 | 0 | 0 | 0 | 0 | 0 | 0 | 0 | 0 | 0 | 0 | 0.0 |
| 44 | 76 3053 | H | F | 0 | 0 | 0 | 0 | Dc | Dc | Dc | Dc | Dc | Dc | Dc | N/A |
| 45 | 76 3479 | C | F | 0 | 0 | 0 | 0 | 0 | 0 | 0 | 0 | 0 | 0 | 0 | 0.0 |
| 46 | 76 8434 | C | F | 0 | 0 | 0 | 0 | 0 | 0 | 0 | 0 | 0 | 0 | 0 | 0.0 |
| 47 | 78 8260 | A | F | 0 | 0 | 0 | 0 | 0 | 0 | 0 | 0 | 0 | 0 | 0 | 0.0 |
| 48 | 80 0847 | C | F | 0 | 0 | 0 | 0 | 0 | 0 | 0 | 0 | 0 | 0 | 0 | 0.0 |
| 49 | 80 7035 | A | M | 0 | 0 | 0 | 0 | 0 | 0 | 0 | 0 | 0 | 0 | 0 | 0.0 |
| 50 | 84 3029 | C | F | 0 | 0 | 0 | 0 | 0 | 0 | 0 | 0 | 0 | 0 | 0 | 0.0 |
| 51 | 92 7220 | C | M | 0 | 0 | 0 | 2 | 3 | D | D | D | D | 0 | 0 | 5.0 |
| 52 | 95 0798 | C | F | 0 | 0 | 0 | 0 | 0 | 0 | 0 | 0 | 0 | 0 | 0 | 0.0 |

(Composition A)

| | | | | | | | | | | | | | | | |
|---|---|---|---|---|---|---|---|---|---|---|---|---|---|---|---|
| 1 | 35 8496 | H | M | 0 | 0 | 0 | 0 | 0 | 0 | 0 | 0 | 0 | 0 | 0 | 0.0 |
| 2 | 44 7255 | C | F | 0 | 0 | 0 | 0 | 0 | 0 | 0 | 0 | 0 | 0 | 0 | 0.0 |
| 3 | 44 9509 | C | F | 0 | 0 | 0 | 0 | 0 | 0 | 0 | 0 | 0 | 0 | 0 | 0.0 |
| 4 | 48 2063 | C | F | 0 | 0 | 0 | 0 | 0 | 0 | 0 | 0 | 0 | 0 | 0 | 0.0 |
| 5 | 48 6033 | H | M | 0 | 0 | 0 | 0 | 0 | 0 | 0 | 0 | 0 | 0 | 0 | 0.0 |
| 6 | 50 1289 | C | F | 0 | 0 | 0 | 0 | 0 | 0 | 0 | 0 | 0 | 0 | 0 | 0.0 |
| 7 | 52 0525 | C | F | 0 | 0 | 0 | 0 | 0 | 0 | 0 | 0 | 0 | 0 | 0 | 0.0 |
| 8 | 52 2026 | H | M | 0 | 0 | 0 | 0 | 0 | 0 | 0 | 0 | 0 | 0 | 0 | 0.0 |
| 9 | 54 0346 | C | F | 0 | 0 | 0 | 0 | 0 | 0 | 0 | 0 | 0 | 0 | 0 | 0.0 |
| 10 | 54 1758 | C | F | 0 | 0 | 0 | 0 | 0 | 0 | 0 | 0 | 0 | 0 | 0 | 0.0 |
| 11 | 54 4281 | C | M | 0 | 0 | 0 | 0 | 0 | 0 | 0 | 0 | 0 | 0 | 0 | 0.0 |
| 12 | 54 4323 | C | F | 0 | 0 | 0 | 0 | 0 | 0 | 0 | 0 | 0 | 0 | 0 | 0.0 |
| 13 | 54 7647 | C | F | 0 | 0 | 0 | 0 | 0 | 0 | 0 | 0 | 0 | 0 | 0 | 0.0 |
| 14 | 56 1236 | C | F | 0 | 0 | 0 | 0 | 0 | 0 | 0 | 0 | 0 | 0 | 0 | 0.0 |
| 15 | 56 1830 | C | F | 0 | 0 | 0 | 0 | 0 | 0 | 0 | 0 | 0 | 0 | 0 | 0.0 |
| 16 | 56 3141 | H | F | 0 | 0 | 0 | 0 | 0 | 0 | 0 | 0 | 0 | 0 | 0 | 0.0 |
| 17 | 56 3379 | C | F | 0 | 0 | 0 | 0 | 0 | 0 | 0 | 0 | 0 | 0 | 0 | 0.0 |
| 18 | 56 3465 | H | F | 0 | 0 | 0 | 0 | 0 | 0 | 0 | 0 | 0 | 0 | 0 | 0.0 |
| 19 | 56 4283 | H | F | 0 | 0 | 0 | 0 | 0 | 0 | 0 | 0 | 0 | 0 | 0 | 0.0 |
| 20 | 56 5529 | C | F | 0 | 0 | 0 | 0 | 0 | 0 | 0 | 0 | 0 | 0 | 0 | 0.0 |
| 21 | 56 6301 | C | F | 0 | 0 | 0 | 0 | 0 | 0 | 0 | 0 | 0 | 0 | 0 | 0.0 |
| 22 | 56 6648 | C | F | 0 | 0 | 0 | 0 | 0 | 0 | 0 | 0 | 0 | 0 | 0 | 0.0 |
| 23 | 56 8344 | H | F | 0 | 0 | 0 | 0 | 0 | 0 | 0 | 0 | 0 | 0 | 0 | 0.0 |
| 24 | 58 8233 | H | F | 0 | 0 | 0 | 0 | 0 | 0 | 0 | 0 | 0 | 0 | 0 | 0.0 |
| 25 | 60 8701 | C | F | 0 | 0 | 0 | 0 | 0 | 0 | 0 | 0 | 0 | 0 | 0 | 0.0 |
| 26 | 60 9466 | C | F | 0 | 0 | 0 | 0 | 0 | 0 | 0 | 0 | 0 | 0 | 0 | 0.0 |
| 27 | 62 1419 | C | F | 0 | 0 | 0 | 0 | 0 | 0 | 0 | 0 | 0 | 0 | 0 | 0.0 |
| 28 | 62 2435 | C | F | 0 | 0 | 0 | 0 | 0 | 0 | 0 | 0 | 0 | 0 | 0 | 0.0 |
| 29 | 62 8070 | H | F | 0 | 0 | 0 | 0 | 0 | 0 | 0 | 0 | 0 | 0 | 0 | 0.0 |
| 30 | 62 9431 | C | F | 0 | 0 | 0 | 0 | 0 | 0 | 0 | 0 | 0 | 0 | 0 | 0.0 |
| 31 | 64 0667 | C | F | 0 | 0 | 0 | 0 | 0 | 0 | 0 | 0 | 0 | 0 | 0 | 0.0 |
| 32 | 64 4506 | C | F | 0 | 0 | 0 | 0 | 0 | 0 | 0 | 0 | 0 | 0 | 0 | 0.0 |
| 33 | 64 4610 | C | F | 0 | 0 | 0 | 0 | 0 | 0 | 0 | 0 | 0 | 0 | 0 | 0.0 |
| 34 | 66 0508 | C | M | 0 | 0 | 0 | 0 | 0 | 0 | 0 | 0 | 0 | 0 | 0 | 0.0 |
| 35 | 66 8456 | C | F | 0 | 0 | 0 | 0 | 0 | 0 | 0 | 0 | 0 | 0 | 0 | 0.0 |
| 36 | 68 6060 | C | F | 0 | 0 | 0 | 0 | 0 | 0 | 0 | 0 | 0 | 0 | 0 | 0.0 |
| 37 | 70 6695 | C | F | 0 | 0 | Dc | Dc | Dc | Dc | Dc | Dc | Dc | Dc | Dc | N/A |
| 38 | 70 9766 | C | F | 0 | 0 | 0 | 0 | 0 | 0 | 0 | 0 | 0 | 0 | 0 | 0.0 |
| 39 | 72 3555 | C | F | 0 | 0 | 0 | 0 | 0 | 0 | 0 | 0 | 0 | 0 | 0 | 0.0 |
| 40 | 72 3637 | H | F | 0 | 0 | 0 | 0 | 0 | 0 | 0 | 0 | 0 | 0 | 0 | 0.0 |
| 41 | 72 4644 | C | F | 0 | 0 | 0 | 0 | 0 | 0 | 0 | 0 | 0 | 0 | 0 | 0.0 |
| 42 | 72 9050 | C | M | 0 | 0 | 0 | 0 | 0 | 0 | 0 | 0 | 0 | 0 | 0 | 0.0 |
| 43 | 73 6193 | H | F | 0 | 0 | 0 | 0 | 0 | 0 | 0 | 0 | 0 | 0 | 0 | 0.0 |
| 44 | 76 3053 | H | F | 0 | 0 | 0 | 0 | Dc | Dc | Dc | Dc | Dc | Dc | Dc | N/A |
| 45 | 76 3479 | C | F | 0 | 0 | 0 | 0 | 0 | 0 | 0 | 0 | 0 | 0 | 0 | 0.0 |
| 46 | 76 8434 | C | F | 0 | 0 | 0 | 0 | 0 | 0 | 0 | 0 | 0 | 0 | 0 | 0.0 |
| 47 | 78 8260 | A | F | 0 | 0 | 0 | 0 | 0 | 0 | 0 | 0 | 0 | 0 | 0 | 0.0 |
| 48 | 80 0847 | C | F | 0 | 0 | 0 | 0 | 0 | 0 | 0 | 0 | 0 | 0 | 0 | 0.0 |
| 49 | 80 7035 | A | M | 0 | 0 | 0 | 0 | 0 | 0 | 0 | 0 | 0 | 0 | 0 | 0.0 |
| 50 | 84 3029 | C | F | 0 | 0 | 0 | 0 | 0 | 0 | 0 | 0 | 0 | 0 | 0 | 0.0 |
| 51 | 92 7220 | C | M | 0 | 0 | 0 | 0 | 0 | 0 | 0 | 0 | 0 | 0 | 0 | 0.0 |
| 52 | 95 0798 | C | F | 0 | 0 | 0 | 0 | 0 | 0 | 0 | 0 | 0 | 0 | 0 | 0.0 |

### (Control)

The scoring scale and definition of symbols in Table 25 is as follows:
0—No evidence of any effect
?—(Barely perceptible) minimal faint (light pink) uniform or spotty erythema
1—(Mild) pink uniform erythema covering most of contact site
2—(Moderate) pink/red erythema visibly uniform in entire contact area
3—(Marked) bright red erythema with accompanying edema, petechiae or papules
4—(Severe) deep red erythema with vesiculation or weeping with or without edema
D—Patch eliminated due to reaction
Dc—Discontinued due to absence of subject on application date
M—Patch applied to an adjacent site after strong test reaction
N/A—Score is not calculated for subjects discontinued before challenge
S—Skin stained from pigment in product
T—Tan

### Example 12: Nail Permeation Study

The primary objective of this study was to determine the rate and extent of in vitro nail permeation of geranic acid and choline across a human nail substrate. Composition A was applied to the nail substrate. Flux was measured over a period of 32 days after application of the formulations. At the end of 32 days, choline and geranic acid were extracted from the nail using DMSO. Test #1 was performed on 5 nails from one donor, whereas Test #2 was performed on two nails from a separate donor.

Transdermal flux and nail retention on a cell by cell basis was provided for both choline and geranic acid. A Dixon's Qtest with 95% confidence was run on the data sets and any data identified as an outlier using this statistical test was not included in the analysis.

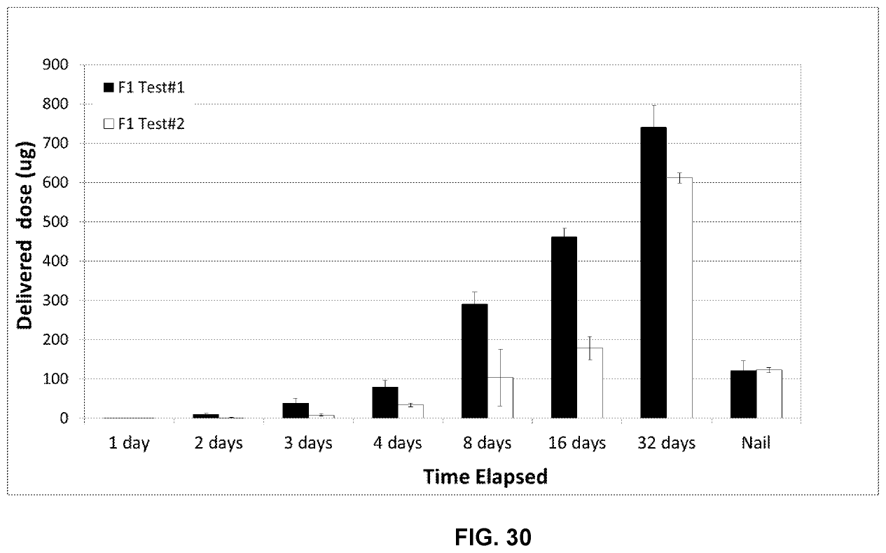

The accumulated dose of choline at each of the time points is shown in Table 26 and FIG. 30.

TABLE 26
Total accumulated dose of choline delivered
over time and corresponding standard error

| | Accumulated dose in μg | | Standard Error (StdErr) | |
|---|---|---|---|---|
| | F1 Test | F1 Test | StdErr Test | StdErr Test |
| Time | #1 | #2 | #1 | #2 |
| 1 day | 0.08 | 0.02 | 0.03 | 0.02 |
| 2 days | 8.9 | 0.88 | 3.66 | 0.88 |
| 3 days | 37.73 | 8.6 | 12.88 | 2.42 |
| 4 days | 78.95 | 33.49 | 17.10 | 4.64 |
| 8 days | 290.53 | 103.48 | 30.31 | 71.78 |
| 16 days | 461.09 | 178.54 | 22.57 | 29.67 |
| 32 days | 740.67 | 611.44 | 55.44 | 12.71 |
| Nail | 121.07 | 122.88 | 24.95 | 6.30 |

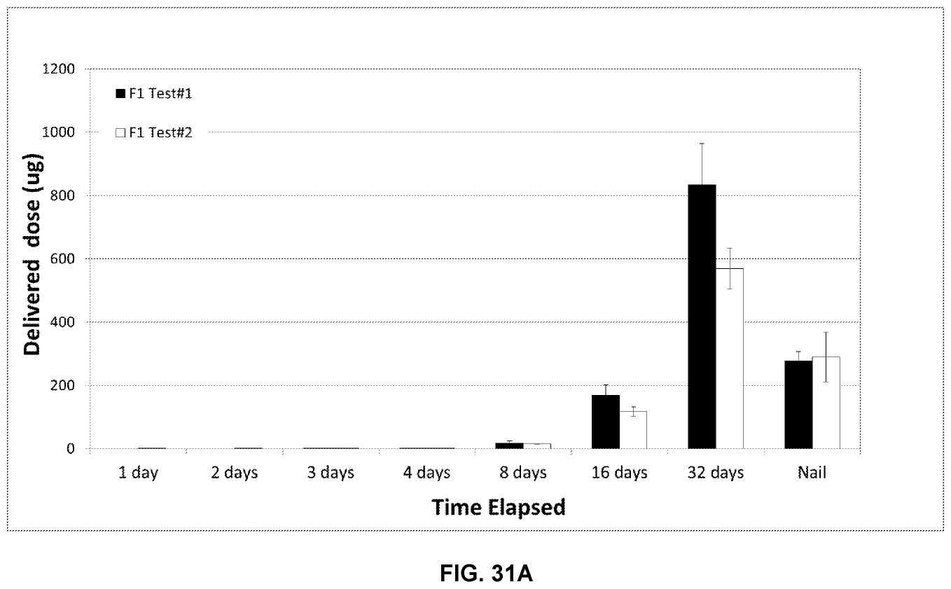

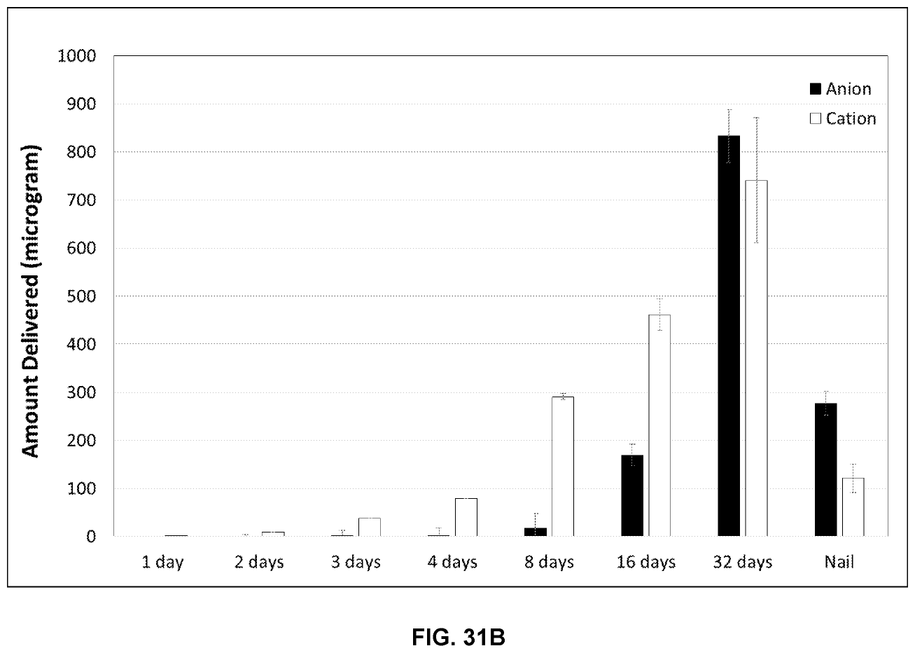

The accumulated dose of geranic acid at each of the time points is shown in Table 27 and FIG. 31A. FIG. 31B shows the amount delivered in microgram of the geranic acid anion (black bars) and the choline cation (white bars) over time.

TABLE 27
Total accumulated dose of geranic acid delivered
over time and corresponding standard error

| | Accumulated dose | | | |
|---|---|---|---|---|
| | in μg/cm² | | Standard Error | |
| | F1 Test | F1 Test | StdErr Test | StdErr Test |
| Time | #1 | #2 | #1 | #2 |
| 1 day | 0.00 | 0.1 | 0.00 | 0.00 |
| 2 days | 0.00 | 0.11 | 0.00 | 0.00 |
| 3 days | 0.04 | 0.1 | 0.02 | 0.02 |
| 4 days | 0.07 | 0.09 | 0.04 | 0.00 |
| 8 days | 17.41 | 14.94 | 6.52 | 0.34 |
| 16 days | 169.29 | 117.36 | 32.62 | 14.67 |
| 32 days | 833.17 | 568.93 | 130.60 | 64.27 |
| Nail | 277.00 | 289.29 | 29.33 | 78.48 |

### Example 13: Appearance and Conductivity of Composition B

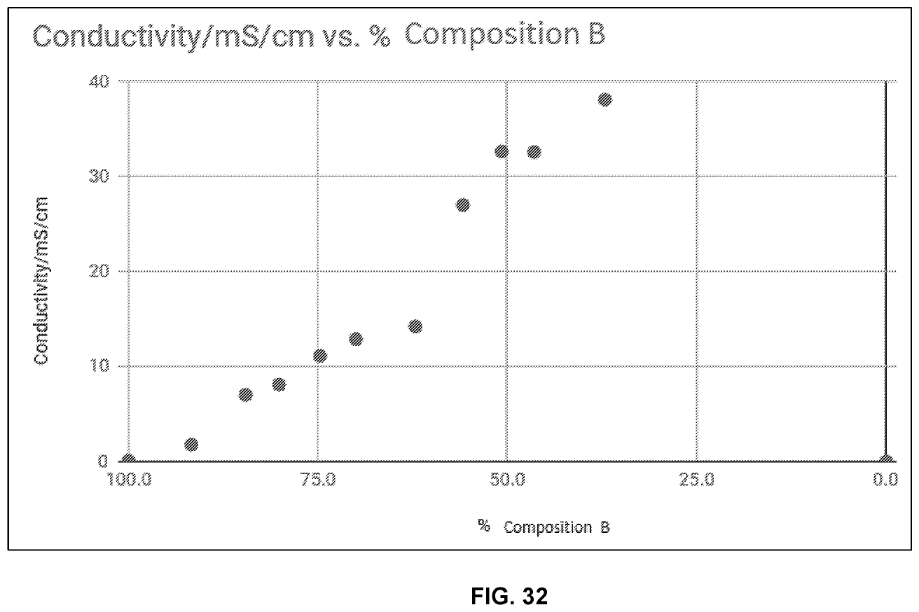

The appearance and conductivity of Composition B in increasing concentrations of Milli-Q® water was investigated. The conductivity is shown in FIG. 32 and the appearance is described in Table 28.

TABLE 28
Appearance of Composition B in varying concentrations of water

| | | % | |
|---|---|---|---|
| Total | Water | Composition | |
| (g) | (g) | B | Appearance and characteristics |
| 13.58 | 0.04 | 93.33 | Thick gel. No milkyness, white precipitation or |
| | | | emulsification observed |
| 13.63 | 0.09 | 86.15 | Still a thick gel. Water slowly mixes into the gel |
| 13.69 | 0.15 | 78.87 | Still thick, but start to see some flow. The added |
| | | | water mixes with the existing gel making it less |
| | | | viscous. No milkyness, precipitation or |
| | | | emulsification. |
| 13.74 | 0.2 | 73.68 | Now a thick liquid, yellowish color lighter color than |
| | | | CAGE. Some gel still stuck at bottom of vial. |
| 13.79 | 0.25 | 69.14 | Thick liquid. No more gel stuck to the bottom of the vial |
| 13.84 | 0.3 | 65.12 | Similar as above, but less viscous |
| 13.9 | 0.36 | 60.87 | Clear liquid, yellowish color |
| 14 | 0.46 | 54.9 | Added water mixes easily, No milkyness, |
| | | | precipitation or emulsification |
| 14.1 | 0.56 | 50 | Same, less viscous |
| 14.31 | 0.77 | 42.11 | Added water mixes easily. No milkyness, |
| | | | precipitation or emulsification. Some foam on top. |
| 14.64 | 1.1 | 33.73 | Same |
| 15.07 | 1.53 | 26.79 | Same |
| 15.7 | 2.16 | 20.59 | Same |
| 17.19 | 3.65 | 13.3 | Same, more foam on top-likely due to mixing |
| 18.63 | 5.09 | 9.91 | Starting to see oil phase when water is added, but |
| | | | mixes in quickly. Clear solution with foam on top. |
| 19.75 | 6.21 | 8.27 | Same |
| 19.99 | 6.45 | 7.99 | White precipitate or phase separation is visible when |
| | | | water is added, but forms a clear solution upon |
| | | | vortexing |
| 21.32 | 7.78 | 6.71 | Milky white emulsion-does not become clear even |
| | | | after vortexing |

The conductivity of Composition B was also examined in increasing concentrations of ethanol. The results are shown in Table 29.

TABLE 29
Conductivity (mS/cm) of Composition B according to pH; the % of
Composition B is shown below, the remaining percentage is ethanol

| Composition | Total | % | | |
|---|---|---|---|---|
| B | Weight | Composition | Conductivity | Appearance and |
| g | g | B | mS/cm | characteristics |
| 1.60 | 2.00 | 80.00 | 1.24 | miscible; clear liquid; takes |
| | | | | vortexing to mix; alcohol |
| | | | | cuts the smell down some |
| 1.60 | 2.35 | 68.10 | 2.83 | clear yellowish liquid; mixes |
| | | | | easily; smell is significantly |
| | | | | reduced |
| 1.60 | 2.72 | 58.80 | 3.96 | clear yellowish liquid; mixes |
| | | | | easily |
| 1.60 | 3.13 | 51.10 | 4.61 | clear yellowish liquid; mixes |
| | | | | easily; can still see the oil |
| | | | | phase as it starts to mix |
| 1.60 | 3.93 | 40.70 | 5.10 | |
| 1.60 | 4.54 | 35.20 | 5.15 | |
| 1.60 | 5.77 | 27.70 | 4.82 | |
| 1.60 | 8.19 | 19.50 | 4.13 | |
| 1.60 | 13.95 | 11.50 | 3.00 | |
| 0.00 | 1.00 | 0.00 | 0.02 | pure ethanol |
| | | | 0.02 | Error (air) |

### Example 14: Study to Evaluate the Safety and Efficacy of Composition A in Rosacea Patients

The study involves two treatment groups, with approximately 20 individuals per treatment group.

Inclusion criteria:
Patient with a clinical diagnosis of moderate to severe facial rosacea
Over 18 years of age
No known medical conditions that interfere with study participation

Exclusion criteria:
Presence of skin diseases at or near the investigational area
Immunosuppressed state or other serious systemic disease
Use of an oral and/or topical medication within 30 days prior to entry into this study
Pregnant or lactating women

Eligible patients are randomized to receive either a 50% Composition A:50% propylene glycol gel base formulation (as described in Table 23) or a placebo, in a blinded fashion. Patients are treated topically twice daily for 12 weeks. Following the screening period and baseline visit, study subjects return at Weeks 3, 6, 9 and 12. A follow up visit takes place at week 16. At each visit, patients are evaluated via lesion count, global assessment tolerability, and safety.

The severity of the overall rosacea condition is measured at baseline and at all follow-up visits. The severity is assessed and graded based on the scales for erythema, telangiectases and number of papulopustular lesions.

### Example 15. Facial Redness, Bumps, and Blemish Study

A study was performed to assess the reduction in facial redness, bumps, and blemishes following use of a gel formulation of Composition A. The study was a 12-week open label study comprising 52 patients.

Inclusion criteria:
Outpatient, male or female of any race, 18 years of age or older. Female subjects of childbearing potential must have a negative UPT at Baseline and practice a reliable method of contraception throughout the study.
Facial redness associated with rosacea with or without bumps or blemishes.
Facial redness (IGA-R) score of 2 or 3 (i.e., mild or moderate).
IGA score of 2 or 3 (i.e., mild or moderate).
Absence of any skin conditions that could interfere with the visual erythema assessments.
Willing to forego any other topical or non-topical treatment, cosmetic, OTC, or prescription on the study areas during treatment (other than sun protection or the study specified face wash and moisturizer).
Willing to use the provided skincare regimen (e.g., face wash and moisturizer) over the duration of the study.
In general good health as determined by medical history and physical examination at the time of screening (Investigator discretion).
Able to follow study instructions and likely to complete all required visits.
Sign the IRB-approved ICF (which includes HIPAA) prior to any study-related procedures being performed.

Exclusion criteria:
Female subjects that are pregnant, breast-feeding, or of childbearing potential and not practicing reliable birth control.
Known hypersensitivity or previous allergic reaction to any constituent of the product (i.e., essential oils, fragrance, choline, phosphatidylcholine, etc.).
Any transient flushing syndrome.
History of basal cell carcinoma within 6 months of Visit 1.
History or presence of a skin condition/disease that is located in the treatment area(s) and might interfere with the diagnosis or evaluation of study parameters (i.e., atopic dermatitis, psoriasis, significant actinic damage, vitiligo, open wounds, infection, etc.).
Diagnosis of severe rosacea, ocular rosacea, rhinophymatous rosacea, or acne fulminans at Baseline.
Blepharitis/meibomianitis requiring systemic treatment by an ophthalmologist.
Uncontrolled systemic disease.
Foreseen unprotected and intense/excessive UV exposure during the course of the study.
Use of any specified concomitant medications/procedures.
Scheduled or planned surgical procedures during the course of the study.
Unable or unwilling to comply with any of the study requirements.
Medical or psychiatric conditions, or a personal situation, that may increase the risk associated with study participation or may interfere with interpretation of study results or compliance of the subject and, in the opinion of the PI, would make the subject inappropriate for study entry.
Clinically significant alcohol or drug abuse, or history of poor cooperation or unreliability.
Exposure to any other investigational drug/device within 30 days prior to study entry.

A gel formulation comprising 40% Composition A (See Examples 2 and 3), 10% propylene glycol, 1% hydroxyethyl cellulose, 1% D-limonene, and 48% water was applied topically to the face (both cheeks, chin, forehead, nose) twice daily after washing and drying of the face. The gel formulation was applied once at night before going to bed and once in the morning using 2-3 drops of the gel. Patients visited the clinical site on weeks 0, 1, 2, 4, 8, and 12.

At each visit, patients were evaluated for primary measurements and secondary measurements. Primary measurements included Investigators Global Assessment on a scale of 0-4 and a count of the number of inflammatory lesions on the face. See Table 30. Secondary measurements included Investigator's Assessment of Redness on a scale of 0-4 and Subject Global Assessment on a scale of 0-4. See Table 31 and Table 32. No concerns with tolerability and no significant adverse events were reported.

TABLE 30
Investigator's Global Assessment (IGA)

| Score | Criteria |
|---|---|
| 0 | Clear skin with no signs of bumps/blemishes |
| 1 | Almost clear; minimal lesions (<5 bumps/blemishes) |
| 2 | Mild facial lesions (6-10 bumps/blemishes) |
| 3 | Moderate lesions; marked redness |
| | (11-25 bumps/blemishes) |
| 4 | Severe lesions; fiery redness (>25 bumps/blemishes) |

TABLE 31
Investigator's Global Assessment of Redness (IGAR)

| Score | Criteria |
|---|---|
| 0 | Clear skin with no signs of redness |
| 1 | Almost clear; slight redness |
| 2 | Mild; definite redness |
| 3 | Moderate; marked redness |
| 4 | Severe; fiery redness |

TABLE 32
Subject Global Assessment

| Score | Criteria |
|---|---|
| 0 | Clear skin (Excellent Effectiveness) |
| 1 | Almost clear skin (Good Effectiveness) |
| 2 | Mild signs and/or symptoms of redness and |
| | bumps/blemishes (Effective) |
| 3 | Moderate signs and/or symptoms of redness |
| | with bumps/blemishes (No significant benefit) |
| 4 | Severe signs and/or symptoms of redness, |
| | bumps/blemishes (No benefit at all) |

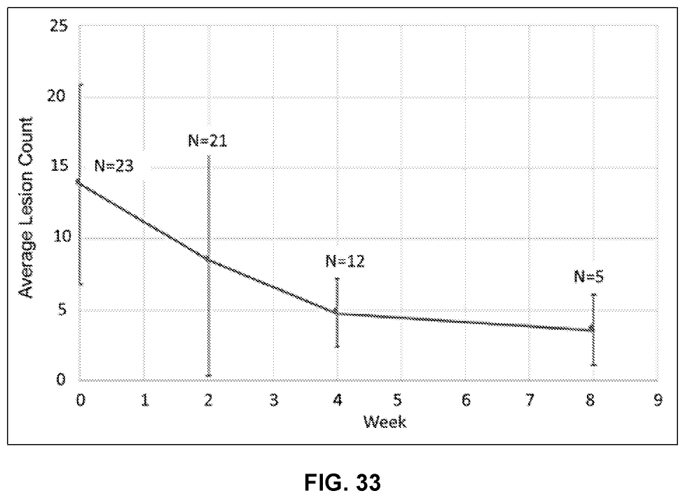

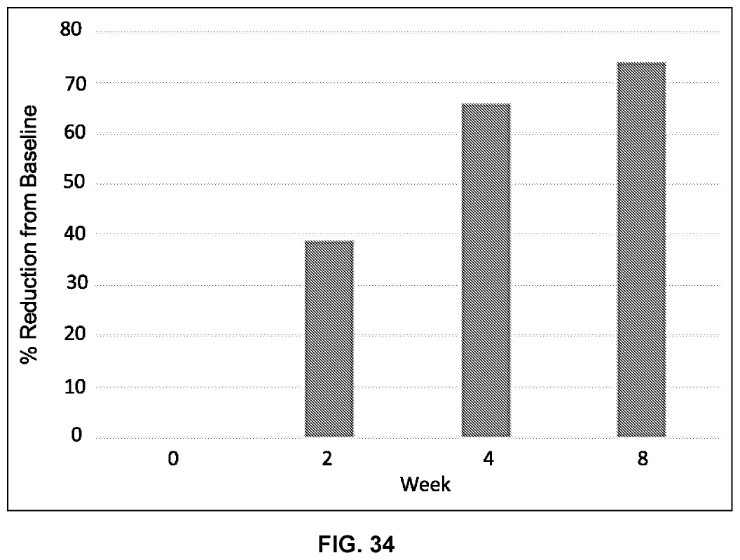

FIG. 33 and FIG. 34 show a significant reduction in inflammatory lesions. FIG. 33 shows a graph of average lesion count (y-axis) over weeks (x-axis). FIG. 34 shows a graph of % reduction from baseline (y-axis) over weeks (x-axis).

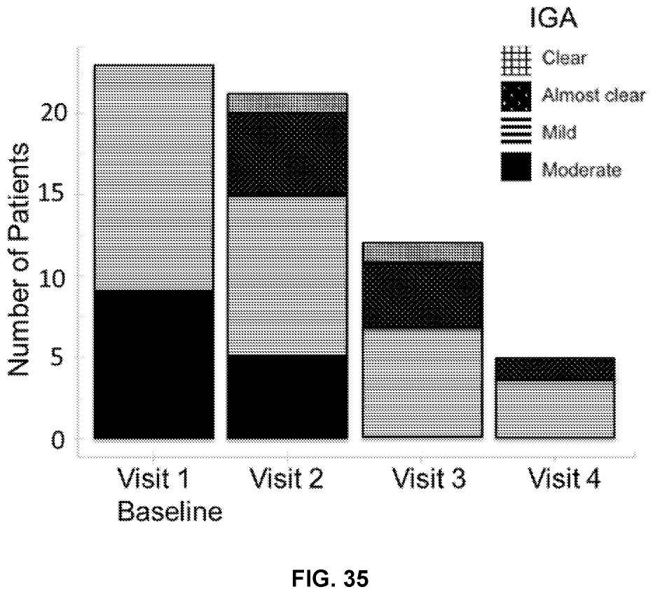

Data from the Investigator's Global Assessment (IGA) demonstrates an improvement in rosacea. See FIG. 35 and Table 33. FIG. 35 shows the number of patients (y-axis) having clear (hatched bars), almost clear (dotted bars), mild (horizontal bars), and moderate (black bars) skin as measured by the IGA. After 4 weeks there was a 41.7% increase in almost clear and clear skin.

TABLE 33
Patients with Almost Clear/Clear Skin

| Week | Almost Clear/Clear |
|---|---|
| 0 | 0% (N = 23) |
| 2 | 28.6% (N = 21) |
| 4 | 41.7% (N = 12) |

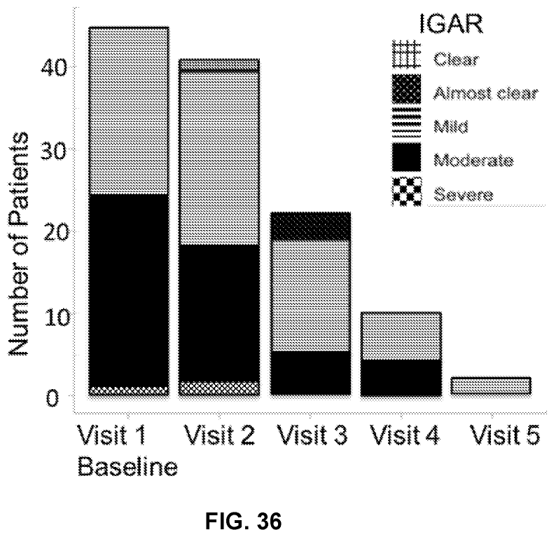

Data from the Investigator's Global Assessment of Redness (IGAR) demonstrates an improvement in rosacea. See FIG. 36 and Table 34. FIG. 36 shows the number of patients (y-axis) having clear (hatched bars), almost clear (dotted bars), mild (horizontal bars), moderate (black bars), and severe (checkered bars) redness as measured by the IGAR. After the third visit, there was a significant reduction in redness.

TABLE 34
Patients with Moderate or Severe Redness

| Visit | Moderate or Severe |
|---|---|
| 1 | 53.3% (N = 45) |
| 2 | 43.9% (N = 41) |
| 3 | 22.7% (N = 22) |

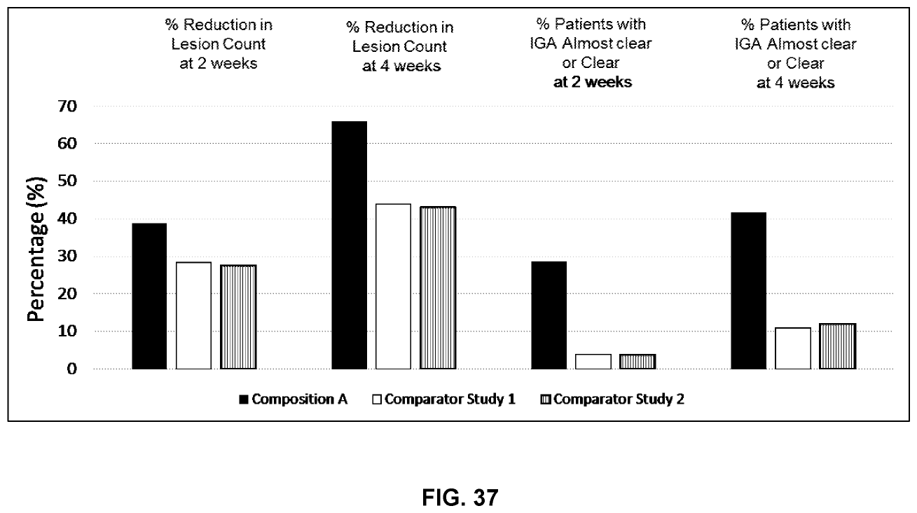

Data from Gel formulation of Composition A were compared against published data from a FDA-approved Comparator in two comparator phase 3 studies (Comparator Study 1 and Comparator Study 2). Composition A as compared to the Comparator resulted in a higher percentage of reduction in lesion count at 2 weeks and 4 weeks (FIG. 37). Further as seen in FIG. 37, there was a higher percentage of patients with almost clear or clear skin as measured by the IGA at 2 weeks and 4 weeks following administration of Composition A as compared to the Comparator.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for treating an inflammatory or infectious skin disease or condition in an individual in need thereof, comprising administering to a skin of the individual a composition comprising:
a. an ionic liquid comprising a choline cation and geranic acid anion; and
b. a pharmaceutically acceptable solvent.

2. The method of claim 1, wherein the inflammatory or infectious skin disease or condition is rosacea, molluscum contagiosum, or onychomycosis.

3. The method of claim 1, wherein the inflammatory or infectious skin disease or condition is rosacea.

4. The method of claim 1, wherein the skin disease or condition is an inflammatory skin disease or condition.

5. The method of claim 1, wherein the pharmaceutically acceptable solvent is selected from the group consisting of: water, ethanol, diisopropyl adipate, polyethylene glycol (PEG), glycerin, propylene glycol, and a combination thereof.

6. The method of claim 1, wherein the composition further comprises a gelling agent.

7. The method of claim 6, wherein the gelling agent is selected from the group consisting of: hydroxyethyl cellulose (HEC), hydroxypropyl cellulose (HPC), hydroxypropylmethyl cellulose (HPMC), and a combination thereof.

8. The method of claim 1, wherein the ionic liquid comprises the choline cation and geranic acid anion in a molar ratio in a range of 1:1 to 1:4 of choline cation to geranic acid anion.

9. The method of claim 1, wherein the ionic liquid comprises the choline cation and geranic acid anion in a molar ratio of 1:1, 1:2, 1:3, or 1:4 of choline cation to geranic acid anion.

10. The method of claim 1, wherein the composition provides an increased antimicrobial action compared to an antimicrobial action of choline or an antimicrobial action of geranic acid.

11. The method of claim 10, wherein the increased antimicrobial action is a 10 fold less concentration of the composition required for complete killing of a microbe relative to a concentration of choline or a concentration of geranic acid required for complete killing of the microbe.

12. The method of claim 1, wherein the composition provides an increased skin permeation relative to a skin permeation of choline or a skin permeation of geranic acid.

13. The method of claim 1, wherein the composition provides an increased conductivity relative to a conductivity of geranic acid and a decreased conductivity relative to a conductivity of choline.

14. The method of claim 1, wherein the ionic liquid is present at a concentration of about 0.1% to 99% of the composition, and the pharmaceutically acceptable solvent comprises a concentration of about 1% to about 99.9% of the composition.

15. The method of claim 1, wherein the composition is formulated for transdermal administration.

16. The method of claim 1, wherein the composition further comprises an additional therapeutic agent selected from the group consisting of: a small molecule drug, an antimicrobial agent, a protein, a peptide, an antibody, a nucleic acid, a chemotherapy agent, and a combination thereof.

17. The method of claim 1, wherein the composition is formulated as a gel, lotion, cream, ointment, solution, or a patch.

18. The method of claim 1, wherein erythema of the skin of the individual is reduced.

19. The method of claim 1, wherein redness of the skin is reduced.

20. The method of claim 1, wherein inflammation of the skin of the individual is reduced.

21. The method of claim 1, wherein a number of lesions on the skin is reduced.

22. The method of claim 1, wherein lesions on the skin of the individual are reduced.

23. A method for treating rosacea in an individual in need thereof, comprising administering to a skin of the individual a composition comprising:
a. an ionic liquid comprising a choline cation and a geranic acid anion; and
b. a pharmaceutically acceptable solvent selected from the group consisting of:
diisopropyl adipate, polyethylene glycol (PEG), glycerin, propylene glycol, and a combination thereof.

24. A method for treating rosacea in an individual in need thereof, comprising administering to a skin of the individual a composition comprising:
a. an ionic liquid comprising a choline cation and a geranic acid anion;
b. a pharmaceutically acceptable solvent selected from the group consisting of:
water, ethanol, diisopropyl adipate, polyethylene glycol (PEG), glycerin, propylene glycol, and a combination thereof; and
c. a gelling agent.

25. The method of claim 23 or claim 24, wherein the rosacea is caused by a mite, bacteria, or a combination thereof.

26. The method of 25, wherein the composition does not induce development of resistance in the mite or the bacteria.

27. The method of claim 23 or claim 24, wherein erythema of the skin of the individual is reduced.

28. The method of claim 23 or claim 24, wherein redness of the skin is reduced.

29. The method of claim 23 or claim 24, wherein inflammation of the skin of the individual is reduced.

30. The method of claim 23 or claim 24, wherein a number of lesions on the skin is reduced.

31. The method of claim 23 or claim 24, wherein lesions on the skin of the individual are reduced.

32. The method of claim 23 or claim 24, wherein the ionic liquid comprises the choline cation and geranic acid anion in a range of 1:1 to 1:4 of choline cation to geranic acid.

33. The method of claim 23 or claim 24, wherein the ionic liquid comprises the choline cation and geranic acid anion in a molar ratio of 1:1, 1:2, 1:3, or 1:4 of choline cation to geranic acid anion.

34. The method of claim 23 or claim 24, wherein the composition provides an increased antimicrobial action compared to an antimicrobial action of choline or an antimicrobial action of geranic acid.

35. The method of claim 34, wherein the increased antimicrobial action is a 10 fold less concentration of the composition required for complete killing of a microbe relative to a concentration of choline or a concentration of geranic acid required for complete killing of the microbe.

36. The method of claim 23 or claim 24, wherein the composition provides an increased skin permeation relative to a skin permeation of choline or a skin permeation of geranic acid.

37. The method of claim 23 or claim 24, wherein the composition provides an increased conductivity relative to a conductivity of geranic acid and a decreased conductivity relative to a conductivity of choline.

38. The method of claim 23 or claim 24, wherein the ionic liquid is present a concentration of about 0.1% to 99% of the composition, and the pharmaceutically acceptable solvent comprises a concentration of about 1% to about 99.9% of the composition.

39. The method of claim 23 or claim 24, wherein the composition is formulated for transdermal administration.

40. The method of claim 23 or claim 24, further comprising an additional therapeutic agent selected from the group consisting of: a small molecule drug, an antimicrobial agent, a protein, a peptide, an antibody, a nucleic acid, a chemotherapy agent, and a combination thereof.

41. The method of claim 23, wherein the composition is formulated as a gel, lotion, cream, ointment, solution, or a patch.

42. The method of claim 24, wherein the gelling agent is selected from the group consisting of: hydroxyethyl cellulose (HEC), hydroxypropyl cellulose (HPC), hydroxypropylmethyl cellulose (HPMC), and a combination thereof.

43. The method of claim 23 or claim 24, further comprising a fragrance agent.

44. The method of claim 43, wherein the fragrance agent is an acid or a terpene of a citrus fruit.

45. The method of claim 44, wherein the citrus fruit is an orange, a grapefruit, a lime, or a lemon.

46. The method of claim 44, wherein the terpene is D-limonene.

47. The method of claim 44, wherein the acid is citric acid or a derivative thereof.

48. A composition comprising:
a. an ionic liquid comprising a choline cation and a geranic acid anion; and
b. a pharmaceutically acceptable solvent selected from the group consisting of:
diisopropyl adipate, polyethylene glycol (PEG), glycerin, propylene glycol, and a combination thereof.

49. A composition comprising:
a. an ionic liquid comprising a choline cation and a geranic acid anion;
b. a pharmaceutically acceptable solvent selected from the group consisting of:
water, ethanol, diisopropyl adipate, polyethylene glycol (PEG), glycerin, propylene glycol, and a combination thereof; and
c. a gelling agent.

50. The composition of claim 48 or claim 49, wherein the ionic liquid comprises the choline cation and geranic acid anion in a molar ratio in a range of 1:1 to 1:4 of choline cation to geranic acid anion.

51. The composition of claim 48 or claim 49, wherein the ionic liquid comprises the choline cation and geranic acid anion in a molar ratio of 1:1, 1:2, 1:3, or 1:4 of choline cation to geranic acid anion.

52. The composition of claim 48 or claim 49, wherein the composition provides an increased antimicrobial action compared to an antimicrobial action of choline or an antimicrobial action of geranic acid.

53. The composition of claim 52, wherein the increased antimicrobial action is a 10 fold less concentration of the composition required for complete killing of a microbe relative to a concentration of choline or a concentration of geranic acid required for complete killing of the microbe.

54. The composition of claim 48 or claim 49, wherein the composition provides an increased skin permeation relative to a skin permeation of choline or a skin permeation of geranic acid.

55. The composition of claim 48 or claim 49, wherein the composition provides an increased conductivity relative to a conductivity of geranic acid and a decreased conductivity relative to a conductivity of choline.

56. The composition of claim 48 or claim 49, wherein the ionic liquid comprises a concentration of about 0.1% to 99% of the composition, and the pharmaceutically acceptable solvent comprises a concentration of about 1% to about 99.9% of the concentration.

57. The composition of claim 48 or claim 49, wherein the composition is formulated for transdermal administration.

58. The composition of claim 48 or claim 49, further comprising an additional therapeutic agent selected from the group consisting of: a small molecule drug, an antimicrobial agent, a protein, a peptide, an antibody, a nucleic acid, a chemotherapy agent, and a combination thereof.

59. The composition of claim 48, wherein the composition is formulated as a gel, lotion, cream, ointment, solution, or a patch.

60. The composition of claim 49, wherein the gelling agent is selected from the group consisting of: hydroxyethyl cellulose (HEC), hydroxypropyl cellulose (HPC), hydroxypropylmethyl cellulose (HPMC), and a combination thereof.

61. The composition of claim 48 or claim 49, further comprising a fragrance agent.

62. The composition of claim 61, wherein the fragrance agent is an acid or a terpene of a citrus fruit.

63. The composition of claim 62, wherein the citrus fruit is an orange, a grapefruit, a lime, or a lemon.

64. The composition of claim 62, wherein the terpene is D-limonene.

65. The composition of claim 62, wherein the acid is citric acid or a derivative thereof.

66. A composition comprising 20% to 60% of an ionic liquid comprising a choline cation and a geranic acid anion, 5% to 20% propylene glycol, and a remaining balance of water.

67. The composition of claim 66 comprising 30% to 50% of the ionic liquid.

68. The composition of claim 66, wherein a molar ratio of the choline cation and geranic acid anion is 1:2.

69. The composition of claim 66 comprising 10% to 15% propylene glycol.

70. The composition of claim 66, wherein the composition further comprises 0.5% to 5% hydroxyethyl cellulose.

71. The composition of claim 66, wherein the composition further comprises 0.5% to 5% D-limonene.

72. The composition of claim 66, wherein the composition is formulated as a gel.

73. The composition of claim 66, wherein the composition is formulated for topical administration.

74. The composition of claim 66, wherein the composition is formulated for twice daily administration.
